(19) United States
(12) Patent Application Publication
(10) Pub. No.: US 2001/0044939 A1
(43) Pub. Date: Nov. 22, 2001

(54) Small subunit of plant acetolactate synthase

(76) Inventors: Lynn M. Abell, Wilmington, DE (US); Saverio Carl Falco, Arden, DE (US); Omolayo O. Famodu, Newark, DE (US)

Correspondence Address: E I DU PONT DE NEMOURS AND COMPANY, LEGAL DEPARTMENT - PATENTS, 1007 MARKET STREET, WILMINGTON, DE, 19898, US

(21) Appl. No.: 09/732,618

(22) Filed: Dec. 8, 2000

Related U.S. Application Data
(60) Provisional application No. 60/174,437, filed on Jan. 4, 2000.

(51) Int. Cl. C12N 15/82; A01H 5/00; C07H 21/04; C12N 15/29; C12N 9/00; C12N 5/04

(52) U.S. Cl. 800/278; 435/183; 536/23.2; 536/23.6; 435/69.1; 435/419

(57) ABSTRACT

This invention relates to an isolated nucleic acid fragment encoding an acetolactate synthase small subunit. The invention also relates to the construction of a chimeric gene encoding all or a portion of the acetolactate synthase small subunit, in sense or antisense orientation, wherein expression of the chimeric gene results in production of altered levels of the acetolactate synthase small subunit in a transformed host cell.

This application claims the benefit of U.S. Provisional Application No. 60/174,437, filed Jan. 4, 2000.

### FIELD OF THE INVENTION

This invention is in the fields of plant molecular biology and herbicide discovery. Specifically, this invention pertains to nucleic acid fragments encoding the small subunit of plant acetolactate synthase, and use of the encoded protein to aid in the discovery of new herbicides that inhibit plant acetolactate synthase activity.

### BACKGROUND OF THE INVENTION

Acetolactate synthase (ALS; EC 4.1.3.18), also known as acetohydroxy acid synthase, is the first committed step in branched chain amino acid biosynthesis in plants and bacteria. The enzyme is known to be the site of action of the several diverse classes of herbicides including the sulfonylureas [Chaleff, R. S. & Mauvais, C. J., Science 224:1443 1445 (1984); LaRossa, R. A. & Schloss, J. V., J. Biol. Chem. 259:8753-8757 (1984); Ray, T. B., Plant Physiol. 75:827-831 (1984)] and the imidazolinones [Shaner et al., Plant Physiol. 76:545-546 (1984)]. Bacterial ALS has been extensively characterized and is known to exist as three isozymes in E. coli. Each isozyme is a tetramer composed of two identical large subunits of approximately 60,000 Da molecular weight and two identical smaller subunits ranging in molecular weight between 9,000 Da and 17,000 Da depending on the isozyme of ALS [Chipman et al., in Biosynthesis of Branched Chain Amino Acids, eds. Barak, Z., Chipman, D. M. & Schloss, J. V. VCH, Weinheim 243-284 (1990)]. There is a high degree of sequence identity shared among the large subunits in the different isozymes, but very low conservation of sequence is observed when comparing the small subunits. The different isozymes also show various sensitivities to feedback inhibition in the presence of valine, leucine and isoleucine. Only isozyme II is insensitive to feedback inhibition. In the absence of the small subunit, the large subunit is still catalytically active, though at a reduced level and shows no inhibition by effectors such as valine and isoleucine [Weinstock et al., J. of Bacteriology 174: 5560-5566 (1992)]. When the large and small subunits of a given isozyme are expressed separately and then mixed, significant increases in specific activity are observed. In the case of isozyme I, a three- to four-fold increase in specific activity is observed upon mixing, whereas in the case of isozyme III, a thirty- to forty-fold increase is observed. Sensitivity to feedback inhibition is restored upon adding the small subunit to the large subunit. Mixing large and small subunits from different isozymes does not produce increases in specific activity or sensitivity to feedback inhibitors.

The enzyme from plants is much less well characterized. Attempts to purify the enzyme from plant extracts have been hampered by the extreme lability of the enzyme and its low abundance. All attempts to purify the enzyme from plant sources have produced only a single major band on an SDS-PAGE gel which varies in molecular weight from 58,000 Da [Durner, J. & Boger, P. Z. Natiurforsch 43c: 850-856 (1988)] to 65,000-66,000 Da [Muhitch et al., Plant Physiol. 83: 451-456 (1987)]. Attempts to immunoprecipitate the enzyme from plant extracts also resulted in the isolation of a single band at 65,000-66,000 Da molecular weight [Singh, B. K. et al., Proc. Natl. Acad. Sci. USA 88:4572-4576 (1991); Bekkaoui, F. et al., Physiologia Plantarum 88:475-484 (1993)]. In the later case, a second band at 36,000 Da was also identified but sequence analysis showed that the protein belonged to a family of aldolases. Thus, since the mid-1980's when the first attempts were made to purify ALS from plant sources, the question as to whether the plant holoenzyme is composed of large and small subunits, in analogy to the bacterial enzyme, has remained unanswered and a matter of some speculation.

The tobacco acetolactate synthase small subunit sequence disclosed herein is published (WO 98/37206 which published Aug. 27, 1998; Hershey et al. (1999) Plant Mol Biol 40:795-806). Arabidopsis thaliana sequences encoding acetolactate synthase small subunits have likewise been published (WO 00/26390 which published May 11, 2000; Sato et al. (1997) DNA Res 4:215-230; Lin et al. (1999) Nature 402:761-768).

### SUMMARY OF THE INVENTION

cDNA clones have now been discovered and identified as the small subunit of plant ALS based upon the sequence identity shared between the polypeptides encoded by the clones and various bacterial ALS small subunits. A full length clone was obtained from Nicotiana plumbaginifolia, expressed in E. coli, and partially purified. Mixing the putative small subunit from Nicotiana with various large subunits from several sources (Arabidopsis and Nicotiana) increased the specific activity of the large subunit 4-15 fold. This trend is similar to that observed with the bacterial enzyme and confirms, functionally, the identification of the cDNA clone as the small subunit of plant ALS.

Accordingly, the present invention comprises a nucleic acid fragment encoding the small subunit of plant ALS. The invention also comprises a method for expression and purification of the small subunit, its use in preparing plant ALS holoenzyme and the use of the holoenzyme to screen for potentially herbicidal compounds based upon holoenzyme inhibition.

More specifically, this invention pertains to an isolated nucleic acid fragment encoding the small subunit of a plant acetolactate synthase, the fragment comprising a member selected from the group consisting of (a) a nucleotide sequence encoding a polypeptide comprising the amino acid sequence set forth in SEQ ID NO:24; and (b) a nucleotide sequence essentially similar to the nucleotide sequence of (a). Preferred is an isolated nucleic acid fragment wherein the nucleotide sequence is set forth in SEQ ID NO:23.

Another embodiment of the instant invention is a plasmid vector comprising a nucleic acid fragment encoding the small subunit of a plant acetolactate synthase operably linked to at least one suitable regulatory sequence and a transformed host cell comprising the aforementioned plasmid vector.

In yet another embodiment, the instant invention pertains to a method for evaluating at least one compound for its ability to inhibit acetolactate synthase activity, the method comprising the steps of: (a) transforming a host cell with the plasmid vector comprising a nucleic acid fragment encoding the small subunit of a plant acetolactate synthase operably linked to at least one suitable regulatory sequence; (b) facilitating expression of the nucleic acid fragment encoding the small subunit of a plant acetolactate synthase; (c) purifying the small subunit of a plant acetolactate synthase expressed by the transformed host cell; (d) mixing the purified small subunit with the large subunit of a plant acetolactate synthase in a suitable container, thereby forming a plant acetolactate synthase holoenzyme; (e) treating the holoenzyme with a compound to be tested; and (f) comparing the acetolactate synthase activity of the holoenzyme that has been treated with a test compound to the activity of an untreated holoenzyme, thereby selecting compounds with potential for herbicidal activity.

The present invention also relates to an isolated polynucleotide comprising: (a) a first nucleotide sequence encoding a first polypeptide comprising at least 35 or 100 amino acids, wherein the amino acid sequence of the first polypeptide and the amino acid sequence of SEQ ID NO:8 have at least 85%, 90%, or 95% identity based on the Clustal alignment method, (b) a second nucleotide sequence encoding a second polypeptide comprising at least 50 or 100 amino acids, wherein the amino acid sequence of the second polypeptide and the amino acid sequence of SEQ ID NO:24 have at least 70%, 80%, 85%, 90%, or 95% identity based on the Clustal alignment method, (c) a third nucleotide sequence encoding a third polypeptide comprising at least 100 amino acids, wherein the amino acid sequence of the third polypeptide and the amino acid sequence of SEQ ID NO:2, SEQ ID NO:4, SEQ ID NO:20, or SEQ ID NO:22 have at least 80%, 85%, 90%, or 95% identity based on the Clustal alignment method, (d) a fourth nucleotide sequence encoding a fourth polypeptide comprising at least 140 amino acids, wherein the amino acid sequence of the fourth polypeptide and the amino acid sequence of SEQ ID NO:14 have at least 80%, 85%, 90%, or 95% identity based on the Clustal alignment method, (e) a fifth nucleotide sequence encoding a fifth polypeptide comprising at least 150 amino acids, wherein the amino acid sequence of the fifth polypeptide and the amino acid sequence of SEQ ID NO:10 or SEQ ID NO:12 have at least 80%, 85%, 90%, or 95% identity based on the Clustal alignment method, (f) a sixth nucleotide sequence encoding a sixth polypeptide comprising at least 200 amino acids, wherein the amino acid sequence of the sixth polypeptide and the amino acid sequence of SEQ ID NO:6 have at least 80%, 85%, 90%, or 95% identity based on the Clustal alignment method, (g) a seventh nucleotide sequence encoding a seventh polypeptide comprising at least 200 amino acids, wherein the amino acid sequence of the seventh polypeptide and the amino acid sequence of SEQ ID NO:18 have at least 85%, 90%, or 95% identity based on the Clustal alignment method, (h) an eighth nucleotide sequence encoding an eighth polypeptide comprising at least 250 amino acids, wherein the amino acid sequence of the eighth polypeptide and the amino acid sequence of SEQ ID NO:16 have at least 80%, 85%, 90%, or 95% identity based on the Clustal alignment method, or (i) the complement of the first, second, third, fourth, fifth, sixth, seventh, or eighth nucleotide sequence, wherein the complement and the first, second, third, fourth, fifth, sixth, seventh, or eighth nucleotide sequence contain the same number of nucleotides and are 100% complementary. The first polypeptide preferably comprises the amino acid sequence of SEQ ID NO:8, the second polypeptide preferably comprises the amino acid sequence of SEQ ID NO:24, the third polypeptide preferably comprises the amino acid sequence of SEQ ID NO:2, SEQ ID NO:4, SEQ ID NO:20, or SEQ ID NO:22, the fourth polypeptide preferably comprises the amino acid sequence of SEQ ID NO:14, the fifth polypeptide preferably comprises the amino acid sequence of SEQ ID NO:10 or SEQ ID NO:12, the sixth polypeptide preferably comprises the amino acid sequence of SEQ ID NO:6, the seventh polypeptide preferably comprises the amino acid sequence of SEQ ID NO:18, and the eighth polypeptide preferably comprises the amino acid sequence of SEQ ID NO:16. The first nucleotide sequence preferably comprises the nucleotide sequence of SEQ ID NO:7, the second nucleotide sequence preferably comprises the nucleotide sequence of SEQ ID NO:23, the third nucleotide sequence preferably comprises the nucleotide sequence of SEQ ID NO:1, SEQ ID NO:3, SEQ ID NO:19, or SEQ ID NO:21, the fourth nucleotide sequence preferably comprises the nucleotide sequence of SEQ ID NO:13, the fifth nucleotide sequence preferably comprises the nucleotide sequence of SEQ ID NO:9 or SEQ ID NO:11, the sixth nucleotide sequence preferably comprises the nucleotide sequence of SEQ ID NO:5, the seventh nucleotide sequence preferably comprises the nucleotide sequence of SEQ ID NO:17, and the eighth nucleotide sequence preferably comprises the nucleotide sequence of SEQ ID NO:15. The first, second, third, fourth, fifth, sixth, seventh, and eighth polypeptides preferably are acetolactate synthase small subunits.

In another embodiment, the present invention relates to a chimeric gene comprising any of the isolated polynucleotides of the present invention operably linked to a regulatory sequence.

In another embodiment, the present invention relates to a vector comprising any of the isolated polynucleotides of the present invention.

In another embodiment, the present invention relates to an isolated polynucleotide fragment comprising a nucleotide sequence comprised by any of the polynucleotides of the present invention, wherein the nucleotide sequence contains at least 30, 40, or 60 nucleotides.

In another embodiment, the present invention relates to an isolated polypeptide comprising: (a) a first amino acid sequence comprising at least 35 or 100 amino acids, wherein the first amino acid sequence and the amino acid sequence of SEQ ID NO:8 have at least 85%, 90%, or 95% identity based on the Clustal alignment method, (b) a second amino acid sequence comprising at least 50 or 100 amino acids, wherein the second amino acid sequence and the amino acid sequence of SEQ ID NO:24 have at least 70%, 80%, 85%, 90%, or 95% identity based on the Clustal alignment method, (c) a third amino acid sequence comprising at least 100 amino acids, wherein the third amino acid sequence and the amino acid sequence of SEQ ID NO:2, SEQ ID NO:4, SEQ ID NO:20, or SEQ ID NO:22 have at least 80%, 85%, 90%, or 95% identity based on the Clustal alignment method, (d) a fourth amino acid sequence comprising at least 140 amino acids, wherein the fourth amino acid sequence and the amino acid sequence of SEQ ID NO:14 have at least 80%, 85%, 90%, or 95% identity based on the Clustal alignment method, (e) a fifth amino acid sequence comprising at least 150 amino acids, wherein the fifth amino acid sequence and the amino acid sequence of SEQ ID NO:10 or SEQ ID NO:12 have at least 80%, 85%, 90%, or 95% identity based on the Clustal alignment method, (f) a sixth amino acid sequence comprising at least 200 amino acids, wherein the sixth amino acid sequence and the amino acid sequence of SEQ ID NO:6 have at least 80%, 85%, 90%, or 95% identity based on the Clustal alignment method, (g) a seventh amino acid sequence comprising at least 200 amino acids, wherein the seventh amino acid sequence and the amino acid sequence of SEQ ID NO:18 have at least 85%, 90%, or 95% identity based on the Clustal alignment method, or (h) an eighth amino acid sequence comprising at least 250 amino acids, wherein the eighth amino acid sequence and the amino acid sequence of SEQ ID NO:16 have at least 80%, 85%, 90%, or 95% identity based on the Clustal alignment method. The first amino acid sequence preferably comprises the amino acid sequence of SEQ ID NO:8, the second amino acid sequence preferably comprises the amino acid sequence of SEQ ID NO:24, the third amino acid sequence preferably comprises the amino acid sequence of SEQ ID NO:2, SEQ ID NO:4, SEQ ID NO:20, or SEQ ID NO:22, the fourth amino acid sequence preferably comprises the amino acid sequence of SEQ ID NO:14, the fifth amino acid sequence preferably comprises the amino acid sequence of SEQ ID NO:10 or SEQ ID NO:12, the sixth amino acid sequence preferably comprises the amino acid sequence of SEQ ID NO:6, the seventh amino acid sequence preferably comprises the amino acid sequence of SEQ ID NO:18, and the eighth amino acid sequence preferably comprises the amino acid sequence of SEQ ID NO:16. The polypeptide preferably is an acetolactate synthase small subunit.

In another embodiment, the present invention relates to a method for transforming a cell comprising introducing any of the isolated polynucleotides of the present invention into a cell, and the cell transformed by this method. Advantageously, the cell is eukaryotic, e.g., a yeast or plant cell, or prokaryotic, e.g., a bacterium.

In another embodiment, the present invention relates to a method for producing a transgenic plant comprising transforming a plant cell with any of the isolated polynucleotides of the present invention and regenerating a plant from the transformed plant cell, the transgenic plant produced by this method, and the seed obtained from this transgenic plant.

In another embodiment, the present invention relates to a virus, preferably a baculovirus, comprising any of the isolated polynucleotides of the present invention or any of the chimeric genes of the present invention.

In another embodiment, the present invention relates to a method of selecting an isolated polynucleotide that affects the level of expression of an acetolactate synthase small subunit polypeptide or enzyme activity in a host cell, preferably a plant cell, the method comprising the steps of: (a) constructing an isolated polynucleotide of the present invention or an isolated chimeric gene of the present invention; (b) introducing the isolated polynucleotide or the isolated chimeric gene into a host cell; (c) measuring the level of the acetolactate synthase small subunit polypeptide or enzyme activity in the host cell containing the isolated polynucleotide; and (d) comparing the level of the acetolactate synthase small subunit polypeptide or enzyme activity in the host cell containing the isolated polynucleotide with the level of the acetolactate synthase small subunit polypeptide or enzyme activity in the host cell that does not contain the isolated polynucleotide.

In another embodiment, the present invention relates to a method of obtaining a nucleic acid fragment encoding a substantial portion of an acetolactate synthase small subunit polypeptide, preferably a plant acetolactate synthase small subunit polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, 11, 13, 15, 17, 19, 21, and 23, and the complement of such nucleotide sequences; and amplifying a nucleic acid fragment (preferably a cDNA inserted in a cloning vector) using the oligonucleotide primer. The amplified nucleic acid fragment preferably will encode a substantial portion of an acetolactate synthase small subunit amino acid sequence.

In another embodiment, the present invention relates to a method of obtaining a nucleic acid fragment encoding all or a substantial portion of the amino acid sequence encoding an acetolactate synthase small subunit polypeptide comprising the steps of: probing a cDNA or genomic library with an isolated polynucleotide of the present invention; identifying a DNA clone that hybridizes with an isolated polynucleotide of the present invention; isolating the identified DNA clone; and sequencing the cDNA or genomic fragment that comprises the isolated DNA clone.

In another embodiment, the present invention concerns a method for positive selection of a transformed cell comprising: (a) transforming a host cell with the chimeric gene of the present invention or an expression cassette of the present invention; and (b) growing the transformed host cell, preferably a plant cell, such as a monocot or a dicot, under conditions which allow expression of the acetolactate synthase small subunit polynucleotide in an amount sufficient to complement a null mutant to provide a positive selection means.

In another embodiment, the present invention relates to a method of altering the level of expression of an acetolactate synthase small subunit in a host cell comprising: (a) transforming a host cell with a chimeric gene of the present invention; and (b) growing the transformed host cell under conditions that are suitable for expression of the chimeric gene wherein expression of the chimeric gene results in production of altered levels of the acetolactate synthase small subunit in the transformed host cell.

### BRIEF DESCRIPTION OF THE DRAWINGS AND SEQUENCE LISTINGS

The invention can be more fully understood from the following detailed description and the accompanying drawings and Sequence Listing which form a part of this application.

FIG. 1 shows the map of the expression plasmid pTrx-HIS/SSU used to express the Nicotiana plumbaginifolia acetolactate synthase small subunit polypeptide.

FIG. 2 shows the map of the expression plasmid pGEX-SSU used to express the Nicotiana plumbaginifolia acetolactate synthase small subunit polypeptide.

FIG. 3 shows an alignment of the amino acid sequences of acetolactate synthase small subunit encoded by the nucleotide sequences derived from corn clone p0094.csst172ra (SEQ ID NO:10), soybean clone sdc2c.pk001.b10 (SEQ ID NO:18), and Nicotiana plumbaginifolia (NCBI GenBank Identifier (GI) No. 5931761; SEQ ID NO:25). Amino acids which are conserved among all and at least two sequences with an amino acid at that position are indicated with an asterisk (*). Dashes are used by the program to maximize alignment of the sequences.

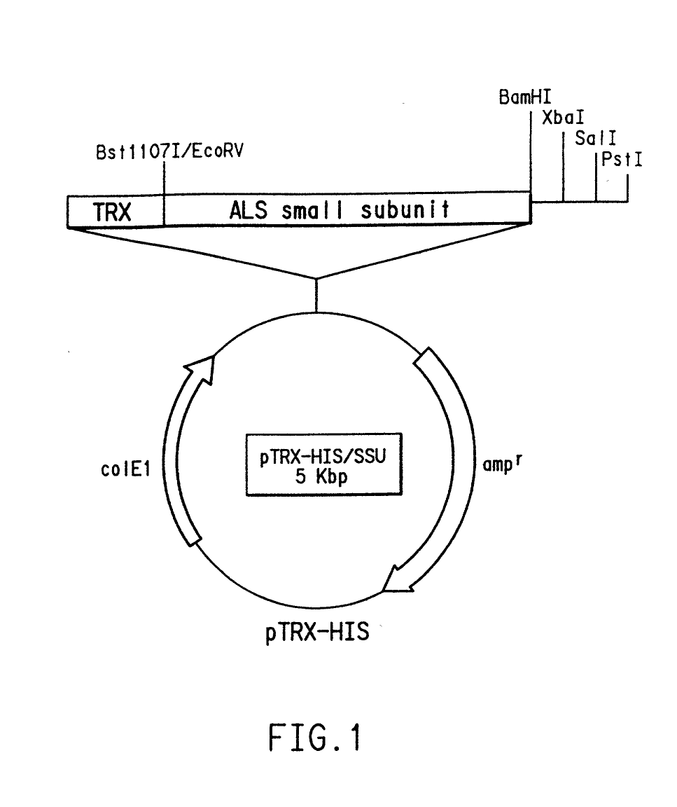

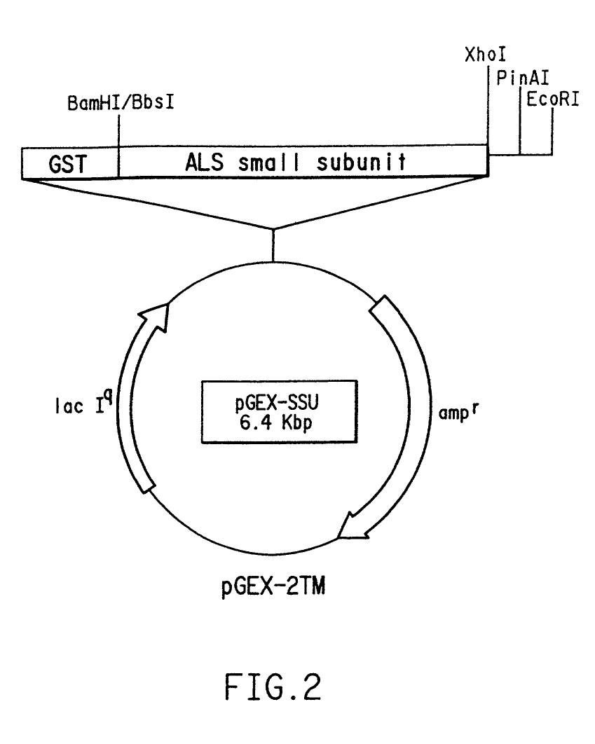

Table 1 lists the polypeptides that are described herein, the designation of the cDNA clones that comprise the nucleic acid fragments encoding polypeptides representing all or a substantial portion of these polypeptides, and the corresponding identifier (SEQ ID NO:) as used in the attached Sequence Listing. Table 1 also identifies the cDNA clones as individual ESTs (“EST”), the sequences of the entire cDNA inserts comprising the indicated cDNA clones (“FIS”), contigs assembled from two or more ESTs (“Contig”), contigs assembled from an FIS and one or more ESTs or PCR fragment sequence (“Contig*”), or sequences encoding the entire protein derived from an FIS, a contig, or an FIS and PCR fragment sequence (“CGS”). Nucleotide SEQ ID NOs:1, 3, 7, 11, 13, 17, and 19 correspond to nucleotide SEQ ID NOs:1, 3, 5, 7, 9, 11, and 13, respectively, presented in U.S. Provisional Application No. 60/174,437, filed Jan. 4, 2000. Amino acid SEQ ID NOs:2, 4, 8, 12, 14, 18, and 20, correspond to amino acid SEQ ID NOs:2, 4, 6, 8, 10, 12, and 14, respectively, presented in U.S. Provisional Application No. 60/174,437, filed Jan. 4, 2000. SEQ ID NOs:23 and 24 were previously presented in Ser. No. 09/377,457 (SEQ ID NO:4), filed on Aug. 19, 1999. SEQ ID NOs:24 and 25 are identical. SEQ ID NO:1 was also previously presented in Ser. No. 09/377,457 (SEQ ID NO:1), filed on Aug. 19, 1999. The sequence descriptions and Sequence Listing attached hereto comply with the rules governing nucleotide and/or amino acid sequence disclosures in patent applications as set forth in 37 C.F.R. §1.821-1.825.

TABLE 1
Acetolactate Synthase (ALS) Small Subunit

| | | | SEQ ID NO: | |
|---|---|---|---|---|
| | Clone | | (Nucleo- | (Amino |
| Protein (Plant Source) | Designation | Status | tide) | Acid) |
| ALS Small Subunit | m15.12.b12.sk20 | EST | 1 | 2 |
| (Corn) | | | | |
| ALS Small Subunit | cen3n.pk0112.c11 | EST | 3 | 4 |
| (Corn) | | | | |
| ALS Small Subunit | cen3n.pk0112.c11 | FIS | 5 | 6 |
| (Corn) | | | | |
| ALS Small Subunit | p0094.csstl72ra | EST | 7 | 8 |
| (Corn) | | | | |
| ALS Small Subunit | p0094.csstl72ra | CGS | 9 | 10 |
| (Corn) | (FIS) | | | |
| ALS Small Subunit | rl0n.pk084.a24 | EST | 11 | 12 |
| (Rice) | | | | |
| ALS Small Subunit | rl0n.pk117.a16 | EST | 13 | 14 |
| (Rice) | | | | |
| ALS Small Subunit | rl0n.pk117.a16 | FIS | 15 | 16 |
| (Rice) | | | | |
| ALS Small Subunit | sdc2c.pk001.b10 | CGS | 17 | 18 |
| (Soybean) | (FIS) | | | |
| ALS Small Subunit | wdk2c.pk015.a13 | EST | 19 | 20 |
| (Wheat) | | | | |
| ALS Small Subunit | wdk2c.pk015.a13 | FIS | 21 | 22 |
| (Wheat) | | | | |
| ALS Small Subunit | pSSU.NP1 (FIS) | CGS | 23 | 24 |
| (Tobacco) | | | | |

SEQ ID NO:25 sets forth the amino acid sequence of an ALS small subunit from Nicotiana plumbaginifolia (NCBI GenBank Identifier (GI) No. 5931761).

SEQ ID NO:26 is the sequence of oligodeoxynucleotide primer SU5R used to prime first strand cDNA synthesis of Nicotiana plumbaginifolia ALS small subunit.

SEQ ID NO:27 is the sequence of oligodeoxynucleotide primer SU4R used for PCR amplification of the single stranded cDNA representing Nicotiana plumbaginifolia ALS small subunit.

SEQ ID NO:28 and SEQ ID NO:29 are oligodeoxynucleotides (pTrx linker1 and pTrx linker2, respectively) that were used to aid construction of the plasmid vector pTrx-Bst1107.

SEQ ID NO:30 and SEQ ID NO:31 are oligodeoxynucleotides (HIS-TAG5 and HIS-TAG3, respectively) that were used to aid construction of the plasmid vector pTrx-HIS.

SEQ ID NO:32 and SEQ ID NO:33 are PCR primers (SSU-PCR1 and SSU-PCR2, respectively) used for amplification of the insert in cDNA clone SSU.NP1.

SEQ ID NO:34 and SEQ ID NO:35 are oligonucleotides (CAM19 and CAM20, respectively) used to modify the plasmid vector pGEX-2T to create the plasmid vector pGEX-2TM.

SEQ ID NO:36 and SEQ ID NO:37 are oligodeoxynucleotides (SSU oligo 9 and SSU oligo 10, respectively) that were used to aid construction of the plasmid vector pGEX-SSU.

SEQ ID NO:38 and SEQ ID NO:39 are oligodeoxynucleotides (SSU oligo 5 and SSU oligo 6, respectively) that were used to aid construction of the plasmid vector pGEX-SSU.

SEQ ID NO:40 and SEQ ID NO:41 are oligodeoxynucleotides (mt704+ and mt800−, respectively) that were used to aid construction of the plasmid vector pMTDRALS.

SEQ ID NO:42 is the full-length cDNA sequence of the Nicotiana plumbaginifolia ALS large subunit contained in the plasmid pALS10.

SEQ ID NO:43 is the predicted amino acid sequence of the polypeptide encoded by the cDNA sequence set forth in SEQ ID NO:42.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### Biological Deposits

The following plasmid has been deposited under the terms of the Budapest Treaty at American Type Culture Collection (ATCC), 12301 Parklawn Drive, Rockville, Md. 20852, and bears the following accession number:

| Plasmid | Accession Number | Date of Deposit |
|---|---|---|
| pSSU.NP1 | ATCC 97876 | February 12, 1997 |

### DETAILED DESCRIPTION OF THE INVENTION

The present invention provides a cDNA sequence encoding the small subunit of plant acetolactate synthase (“ALS”). When plant ALS small subunit protein (“SSU”) is mixed with the gene product referred to by others as plant acetolactate synthase (and referred to herein as the ALS large subunit or “LSU”), the resulting reconstituted ALS holoenzyme shows both increased catalytic efficiency and solution stability when compared to the large subunit alone. The beneficial effects of reconstituting native ALS with this small subunit is not species-specific as shown by the ability of the N. plumbaginifolia small subunit to form an active complex with large subunits from other plant species.

It is expected that divergent plant species will have ALS small subunit genes that encode the same functional protein as that encoded by the cDNA clones described herein. Therefore, it is expected that the invention can also be accomplished using ALS small subunit sequences from other plant species, both monocotyledonous and dicotyledonous. Indeed, based upon the work presented here, it is expected that the invention may be accomplished by mixing small and large ALS subunits from divergent species.

To accomplish the invention, single pass DNA sequence analysis was performed on individual clones from a cDNA library made using RNA from corn embryos that were harvested 15 days post pollination. The sequences were compared to known sequences in the GenBank database until a clone was identified as the small subunit of plant ALS by the similarity of its coding region with those of various bacterial ALS small subunits. A DNA fragment comprising the coding region from this clone was isolated and operably linked to suitable bacterial regulatory sequences to create plasmids capable of directing the expression of plant ALS small subunit in E. coli as thioredoxin (TRX) and glutathione-S-transferase (GST) fusion proteins. These plasmids were introduced separately into suitable strains of E. coli and the TRX- and GST-plant SSU fusion proteins were produced after suitable induction and expression of the chimeric genes encoding the fusion proteins.

The recombinant ALS large and small subunits may be produced using any number of methods by those skilled in the art. Such methods include, but are not limited to, expression in bacteria, eukaryotic cell culture, in planta, and using viral expression systems in suitably infected organisms or cell lines. Large and small ALS subunits may be expressed separately as mature proteins, or may be co-expressed in E. coli or another suitable expression background. In addition, whether expressed separately or in combination, large and small subunits may be expressed either as mature forms of the proteins as observed in vivo or as fusion proteins by covalent attachment to a variety of enzymes, proteins or affinity tags. Common fusion protein partners include glutathione S-transferase (“GST”), thioredoxin (“Trx”), maltose binding protein, and C- and/or N-terminal hexahistidine polypeptide (“(His)₆”). The fusion proteins may be engineered with a protease recognition site at the fusion point so that fusion partners can be separated by protease digestion to yield intact mature large or small subunit. Examples of such proteases include thrombin, enterokinase and factor Xa. However, any protease can be used which specifically cleaves the peptide connecting the fusion protein and either the large or small subunit.

Purification of the ALS subunits or the holoenzyme may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. When the ALS subunits are expressed as fusion proteins, the purification protocol may include the use of an affinity resin which is specific for the fusion protein tag attached to the expressed large or small subunit or an affinity resin containing ligands which are specific for the small subunit. In a preferred embodiment of the invention, small subunit of ALS is expressed as a fusion protein coupled to the C-terminus of thioredoxin. In addition, a (His)₆ peptide may be engineered into the N-terminus of the fused thioredoxin moiety to afford additional opportunities for affinity purification. The preferred purification protocol embodied in the examples involves the use of arsene oxide affinity chromatography with the commercially available ThioBond™ resin (Invitrogen Corporation, San Diego, Calif.) which has affinity for thioredoxin. Other suitable affinity resins could be synthesized by linking the appropriate ligands to any suitable resin such as Sepharose-4B. In a preferred embodiment of the invention, the thioredoxin-SSU fusion protein is eluted using dithiothreitol; however, elution may be accomplished using other reagents which interact to displace the thioredoxin from the resin. These reagents include β-mercaptoethanol or other reduced thiol. The eluted fusion protein may be subjected to further purification by traditional means as stated above, if desired. Proteolytic cleavage of the thioredoxin fusion protein and the ALS small subunit to yield a complete small subunit may be accomplished after the fusion protein is purified, while the protein is still bound to the ThioBond™ affinity resin or other resin, or in the presence of an ALS large subunit.

The generality of this procedure is demonstrated in a preferred embodiment of the invention whereby the small subunit protein is expressed and purified as a fusion to GST. The preferred purification protocol for this fusion protein embodied in the examples involves the use of a glutathione-containing Sepharose- or agarose-based affinity resin which has affinity for GST. Other suitable affinity resins could be synthesized by linking the appropriate ligand to a suitable resin. Moreover, other purification protocols could be developed using conventional chromatography or other protein purification techniques. In a preferred embodiment of the invention, the GST-SSU is eluted using glutathione at pH 9.0; however, elution may be accomplished using other reagents and conditions of pH and ionic strength which serve to weaken the interaction between GST and the glutathione-containing resin.

In a preferred embodiment of the invention, the Arabidopsis ALS large subunit is expressed and purified as a fusion protein with glutathione-S-transferase; however, large subunit may be purified by a number of different means as stated above.

Another preferred embodiment of the invention comprises expression of the Nicotiana plumbaginifolia ALS-LSU as a mature enzyme containing a partial chloroplast transit peptide. As exemplified herein, this enzyme is purified using conventional protein purification procedures. Variations on these procedures using different column materials and conditions can easily be envisioned by the skilled artisan. This enzyme may also be expressed as a fusion protein and affinity-purified as described above.

Acetolactate synthase holoenzyme, which is defined as a combination of large and small subunits, may be prepared by (i) mixing partially or completely purified large and small subunits, (ii) co-purification of the holoenzyme which is either prepared by co-expression or by mixing cell extracts containing individually expressed subunits either as fusion proteins or as mature subunits, or (iii) by mixing purified or partially purified fusion proteins. In the example provided of the invention, the fusion protein of the small subunit is mixed either with a fusion protein of the large subunit or a mature form of the large subunit. In both cases, increases in specific activity were observed upon incubation compared to the large subunit by itself. Activity is further enhanced by the addition of a specific protease, in this case thrombin, to cleave the fusion protein affinity tag to produce mature small and in some cases large subunits. The inclusion of such non-specific proteins such as bovine serum albumin did not produce the same effect as the presence of the small subunit. In the present example, phosphate buffered saline at pH 8 was used. Buffers in the range of pH 6.5 to 9 are preferred with buffers in the range of pH 7 to 7.6 being optimal. Cofactors required for ALS activity may also be present in the holoenzyme reconstitution such as flavin adenine dinucleotide, thiamine pyrophosphate and divalent metal ions. Stabilizing agents such as dithiothreitol and glycerol may also be used.

Acetolactate synthase holoenzyme is sensitive to inhibition by herbicidal compounds known to inhibit ALS in vivo as their mode of action. Thus, the ALS holoenzyme is useful in screening for novel crop protection chemicals.

In the context of this disclosure, a number of terms shall be utilized. The terms “polynucleotide”, “polynucleotide sequence”, “nucleic acid sequence”, and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. An isolated polynucleotide of the present invention may include at least one of 60 contiguous nucleotides, preferably at least one of 40 contiguous nucleotides, most preferably one of at least 30 contiguous nucleotides derived from SEQ ID NOs:1, 3, 5, 7, 9, 11, 13, 15, 17, 19, 21, or 23 or the complement of such sequences.

The term “isolated” polynucleotide refers to a polynucleotide that is substantially free from other nucleic acid sequences, such as other chromosomal and extrachromosomal DNA and RNA, that normally accompany or interact with it as found in its naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is made by an artificial combination of two otherwise separated segments of sequence, e.g., by chemical synthesis or by the manipulation of isolated nucleic acids by genetic engineering techniques.

As used herein, “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to nucleic acid fragments wherein changes in one or more nucleotide bases does not affect the ability of the nucleic acid fragment to mediate alteration of gene expression by gene silencing through for example antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate gene silencing or alteration of the functional properties of the resulting protein molecule. It is therefore understood that the invention encompasses more than the specific exemplary nucleotide or amino acid sequences and includes functional equivalents thereof. The terms “substantially similar” and “corresponding substantially” are used interchangeably herein.

Substantially similar nucleic acid fragments may be selected by screening nucleic acid fragments representing subfragments or modifications of the nucleic acid fragments of the instant invention, wherein one or more nucleotides are substituted, deleted and/or inserted, for their ability to affect the level of the polypeptide encoded by the unmodified nucleic acid fragment in a plant or plant cell. For example, a substantially similar nucleic acid fragment representing at least one of 30 contiguous nucleotides derived from the instant nucleic acid fragment can be constructed and introduced into a plant or plant cell. The level of the polypeptide encoded by the unmodified nucleic acid fragment present in a plant or plant cell exposed to the substantially similar nucleic fragment can then be compared to the level of the polypeptide in a plant or plant cell that is not exposed to the substantially similar nucleic acid fragment.

For example, it is well known in the art that antisense suppression and co-suppression of gene expression may be accomplished using nucleic acid fragments representing less than the entire coding region of a gene, and by using nucleic acid fragments that do not share 100% sequence identity with the gene to be suppressed. Moreover, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Consequently, an isolated polynucleotide comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, 11, 13, 15, 17, 19, 21, and 23 and the complement of such nucleotide sequences may be used in methods of selecting an isolated polynucleotide that affects the expression of an acetolactate synthase small subunit polypeptide in a host cell. A method of selecting an isolated polynucleotide that affects the level of expression of a polypeptide in a virus or in a host cell (eukaryotic, such as plant or yeast, prokaryotic such as bacterial) may comprise the steps of: constructing an isolated polynucleotide of the present invention or an isolated chimeric gene of the present invention; introducing the isolated polynucleotide or the isolated chimeric gene into a host cell; measuring the level of a polypeptide or enzyme activity in the host cell containing the isolated polynucleotide; and comparing the level of a polypeptide or enzyme activity in the host cell containing the isolated polynucleotide with the level of a polypeptide or enzyme activity in a host cell that does not contain the isolated polynucleotide.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C.

Substantially similar nucleic acid fragments of the instant invention may also be characterized by the percent identity of the amino acid sequences that they encode to the amino acid sequences disclosed herein, as determined by algorithms commonly employed by those skilled in this art. Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above identities but typically encode a polypeptide having at least 35 or 50 amino acids, preferably at least 100 amino acids, more preferably at least 140 or 150 amino acids, still more preferably at least 200 amino acids, and most preferably at least 250 amino acids. Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually by one skilled in the art, or by using computer-based sequence comparison and identification tools that employ algorithms such as BLAST (Basic Local Alignment Search Tool; Altschul et al. (1993) J. Mol. Biol. 215:403-410; see also www.ncbi.nlm.nih.gov/BLAST/). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign-gene” refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or may be composed of different elements derived from different promoters found in nature, or may even comprise synthetic nucleotide segments. It is understood by those skilled in the art that different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82. It is further recognized that since in most cases the exact boundaries of regulatory sequences have not been completely defined, nucleic acid fragments of different lengths may have identical promoter activity.

“Translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Mol. Biotechnol. 3:225-236).

“3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into polypeptides by the cell. “cDNA” refers to DNA that is complementary to and derived from an mRNA template. The cDNA can be single-stranded or converted to double stranded form using, for example, the Klenow fragment of DNA polymerase I. “Sense-RNA” refers to an RNA transcript that includes the mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of two or more nucleic acid fragments on a single polynucleotide so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

A “protein” or “polypeptide” is a chain of amino acids arranged in a specific order determined by the coding sequence in a polynucleotide encoding the polypeptide. Each protein or polypeptide has a unique function.

“Altered levels” or “altered expression” refers to the production of gene product(s) in transgenic organisms in amounts or proportions that differ from that of normal or non-transformed organisms.

“Null mutant” refers here to a host cell which either lacks the expression of a certain polypeptide or expresses a polypeptide which is inactive or does not have any detectable expected enzymatic function.

“Mature protein” or the term “mature” when used in describing a protein refers to a post-translationally processed polypeptide; i.e., one from which any pre- or propeptides present in the primary translation product have been removed. “Precursor protein” or the term “precursor” when used in describing a protein refers to the primary product of translation of mRNA; i.e., with pre- and propeptides still present. Pre- and propeptides may be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference). Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Maniatis”).

“PCR” or “polymerase chain reaction” is well known by those skilled in the art as a technique used for the amplification of specific DNA segments (U.S. Pat. Nos. 4,683,195 and 4,800,159).

“Fusion protein” refers to a polypeptide that is produced with additional amino acids at either its N-or C-terminus to aid in its expression and/or purification.

“Inhibition” refers to a decrease in the catalytic activity of an enzyme or holoenzyme complex by reversible or irreversible binding of a chemical to the enzyme.

“Holoenzyme” is defined as an intact enzyme containing all of the subunits and cofactors required for full activity. In the case of a plant acetolactate synthase these include a small subunit comprising a protein of approximately 45,000 Da, a large subunit comprising a protein of approximately 65,000 Da, and cofactors flavin adenine dinucleotide, thiamine pyrophosphate and divalent metal ions.

“Specific activity” is the number of enzyme units per mg protein. “Units” is defined as the micromoles of product produced per minute of reaction time.

The present invention relates to an isolated polynucleotide comprising: (a) a first nucleotide sequence encoding a first polypeptide comprising at least 35 or 100 amino acids, wherein the amino acid sequence of the first polypeptide and the amino acid sequence of SEQ ID NO:8 have at least 85%, 90%, or 95% identity based on the Clustal alignment method, (b) a second nucleotide sequence encoding a second polypeptide comprising at least 50 or 100 amino acids, wherein the amino acid sequence of the second polypeptide and the amino acid sequence of SEQ ID NO:24 have at least 70%, 80%, 85%, 90%, or 95% identity based on the Clustal alignment method, (c) a third nucleotide sequence encoding a third polypeptide comprising at least 100 amino acids, wherein the amino acid sequence of the third polypeptide and the amino acid sequence of SEQ ID NO:2, SEQ ID NO:4, SEQ ID NO:20, or SEQ ID NO:22 have at least 80%, 85%, 90%, or 95% identity based on the Clustal alignment method, (d) a fourth nucleotide sequence encoding a fourth polypeptide comprising at least 140 amino acids, wherein the amino acid sequence of the fourth polypeptide and the amino acid sequence of SEQ ID NO:14 have at least 80%, 85%, 90%, or 95% identity based on the Clustal alignment method, (e) a fifth nucleotide sequence encoding a fifth polypeptide comprising at least 150 amino acids, wherein the amino acid sequence of the fifth polypeptide and the amino acid sequence of SEQ ID NO:10 or SEQ ID NO:12 have at least 80%, 85%, 90%, or 95% identity based on the Clustal alignment method, (f) a sixth nucleotide sequence encoding a sixth polypeptide comprising at least 200 amino acids, wherein the amino acid sequence of the sixth polypeptide and the amino acid sequence of SEQ ID NO:6 have at least 80%, 85%, 90%, or 95% identity based on the Clustal alignment method, (g) a seventh nucleotide sequence encoding a seventh polypeptide comprising at least 200 amino acids, wherein the amino acid sequence of the seventh polypeptide and the amino acid sequence of SEQ ID NO:18 have at least 85%, 90%, or 95% identity based on the Clustal alignment method, (h) an eighth nucleotide sequence encoding an eighth polypeptide comprising at least 250 amino acids, wherein the amino acid sequence of the eighth polypeptide and the amino acid sequence of SEQ ID NO:16 have at least 80%, 85%, 90%, or 95% identity based on the Clustal alignment method, or (i) the complement of the first, second, third, fourth, fifth, sixth, seventh, or eighth nucleotide sequence, wherein the complement and the first, second, third, fourth, fifth, sixth, seventh, or eighth nucleotide sequence contain the same number of nucleotides and are 100% complementary. The first polypeptide preferably comprises the amino acid sequence of SEQ ID NO:8, the second polypeptide preferably comprises the amino acid sequence of SEQ ID NO:24, the third polypeptide preferably comprises the amino acid sequence of SEQ ID NO:2, SEQ ID NO:4, SEQ ID NO:20, or SEQ ID NO:22, the fourth polypeptide preferably comprises the amino acid sequence of SEQ ID NO:14, the fifth polypeptide preferably comprises the amino acid sequence of SEQ ID NO:10 or SEQ ID NO:12, the sixth polypeptide preferably comprises the amino acid sequence of SEQ ID NO:6, the seventh polypeptide preferably comprises the amino acid sequence of SEQ ID NO:18, and the eighth polypeptide preferably comprises the amino acid sequence of SEQ ID NO:16. The first nucleotide sequence preferably comprises the nucleotide sequence of SEQ ID NO:7, the second nucleotide sequence preferably comprises the nucleotide sequence of SEQ ID NO:23, the third nucleotide sequence preferably comprises the nucleotide sequence of SEQ ID NO:1, SEQ ID NO:3, SEQ ID NO:19, or SEQ ID NO:21, the fourth nucleotide sequence preferably comprises the nucleotide sequence of SEQ ID NO:13, the fifth nucleotide sequence preferably comprises the nucleotide sequence of SEQ ID NO:9 or SEQ ID NO:11, the sixth nucleotide sequence preferably comprises the nucleotide sequence of SEQ ID NO:5, the seventh nucleotide sequence preferably comprises the nucleotide sequence of SEQ ID NO:17, and the eighth nucleotide sequence preferably comprises the nucleotide sequence of SEQ ID NO:1 5. The first, second, third, fourth, fifth, sixth, seventh, and eighth polypeptides preferably are acetolactate synthase small subunits.

Nucleic acid fragments encoding at least a portion of several acetolactate synthase small subunits have been isolated and identified by comparison of random plant cDNA sequences to public databases containing nucleotide and protein sequences using the BLAST algorithms well known to those skilled in the art. The nucleic acid fragments of the instant invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other acetolactate synthase small subunit, either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, an entire sequence can be used directly to synthesize DNA probes by methods known to the skilled artisan such as random primer DNA labeling, nick translation, end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al. (1988) Proc. Natl. Acad. Sci. USA 85:8998-9002) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al. (1989) Proc. Natl. Acad. Sci. USA 86:5673-5677; Loh et al. (1989) Science 243:217-220). Products generated by the 3′ and 5′ RACE procedures can be combined to generate full-length cDNAs (Frohman and Martin (1989) Techniques 1:165). Consequently, a polynucleotide comprising a nucleotide sequence of at least one of 60 (preferably one of at least 40, most preferably one of at least 30) contiguous nucleotides derived from a nucleotide sequence selected from the group consisting of SEQ ID NOs:1, 3, 5, 7, 9, 11, 13, 15, 17, 19, 21, and 23 and the complement of such nucleotide sequences may be used in such methods to obtain a nucleic acid fragment encoding a substantial portion of an amino acid sequence of a polypeptide.

The present invention relates to a method of obtaining a nucleic acid fragment encoding a substantial portion of an acetolactate synthase small subunit polypeptide, preferably a substantial portion of a plant acetolactate synthase small subunit polypeptide, comprising the steps of: synthesizing an oligonucleotide primer comprising a nucleotide sequence of at least one of 60 (preferably at least one of 40, most preferably at least one of 30) contiguous nucleotides derived from a nucleotide sequence selected from the group consisting of SEQ ID NOs:1, 3, 5, 7, 9, 11, 13, 15, 17, 19, 21, and 23, and the complement of such nucleotide sequences; and amplifying a nucleic acid fragment (preferably a cDNA inserted in a cloning vector) using the oligonucleotide primer. The amplified nucleic acid fragment preferably will encode a portion of an acetolactate synthase small subunit polypeptide.

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner (1984) Adv. Immunol. 36:1-34; Maniatis).

In another embodiment, this invention concerns viruses and host cells comprising either the chimeric genes of the invention as described herein or an isolated polynucleotide of the invention as described herein. Examples of host cells which can be used to practice the invention include, but are not limited to, yeast, bacteria, and plants.

As was noted above, the nucleic acid fragments of the instant invention may be used to create transgenic plants in which the disclosed polypeptides are present at higher or lower levels than normal or in cell types or developmental stages in which they are not normally found. This would have the effect of altering the level of herbicide resistance in those transgenic plants and transformed cells. Consequently, the level of herbicide resistance in these cells may be used as a dominant selectable marker for plant transformation. For example, plant cells may be transformed with a construct to overexpress acetolactate synthase small subunit along with sulfonylurea-herbicide resistant large subunit; this would have the effect of producing cells that are resistant to sulfonylurea. The transformed cells may thus be grown in sulfonylurea-containing media to select for plant cells that are expessing sufficiently enough the foreign construct.

In addition, the ALS small subunit may be overexpressed in E. coli or other suitable hosts using the nucleic acid fragments disclosed herein, generating sufficient quantities of protein which may be used to reconstitute the holoenzyme for in vitro herbicide discovery assays.

Also, nucleic acid fragments encoding the ALS small subunit may be used to overexpress the active holoenzyme in plants. Overexpression of both the large and small subunits could be another means to genetically engineer herbicide resistance into plants.

Overexpression of the proteins of the instant invention may be accomplished by first constructing a chimeric gene in which the coding region is operably linked to a promoter capable of directing expression of a gene in the desired tissues at the desired stage of development. The chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression.

Plasmid vectors comprising the instant isolated polynucleotide (or chimeric gene) may be constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the instant polypeptides to different cellular compartments, or to facilitate its secretion from the cell. It is thus envisioned that the chimeric gene described above may be further supplemented by directing the coding sequence to encode the instant polypeptides with appropriate intracellular targeting sequences such as transit sequences (Keegstra (1989) Cell 56:247-253), signal sequences or sequences encoding endoplasmic reticulum localization (Chrispeels (1991) Ann. Rev. Plant Phys. Plant Mol. Biol. 42:21-53), or nuclear localization signals (Raikhel (1992) Plant Phys. 100:1627-1632) with or without removing targeting sequences that are already present. While the references cited give examples of each of these, the list is not exhaustive and more targeting signals of use may be discovered in the future.

It may also be desirable to reduce or eliminate expression of genes encoding the instant polypeptides in plants for some applications. In order to accomplish this, a chimeric gene designed for co-suppression of the instant polypeptide can be constructed by linking a gene or gene fragment encoding that polypeptide to plant promoter sequences. Alternatively, a chimeric gene designed to express antisense RNA for all or part of the instant nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated.

Molecular genetic solutions to the generation of plants with altered gene expression have a decided advantage over more traditional plant breeding approaches. Changes in plant phenotypes can be produced by specifically inhibiting expression of one or more genes by antisense inhibition or cosuppression (U.S. Pat. Nos. 5,190,931, 5,107,065 and 5,283,323). An antisense or cosuppression construct would act as a dominant negative regulator of gene activity. While conventional mutations can yield negative regulation of gene activity these effects are most likely recessive. The dominant negative regulation available with a transgenic approach may be advantageous from a breeding perspective. In addition, the ability to restrict the expression of a specific phenotype to the reproductive tissues of the plant by the use of tissue specific promoters may confer agronomic advantages relative to conventional mutations which may have an effect in all tissues in which a mutant gene is ordinarily expressed.

The person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenics for those that most effectively display the desired phenotype. Accordingly, the skilled artisan will develop methods for screening large numbers of transformants. The nature of these screens will generally be chosen on practical grounds. For example, one can screen by looking for changes in gene expression by using antibodies specific for the protein encoded by the gene being suppressed, or one could establish assays that specifically measure enzyme activity. A preferred method will be one which allows large numbers of samples to be processed rapidly, since it will be expected that a large number of transformants will be negative for the desired phenotype.

In another embodiment, the present invention relates to an isolated polypeptide comprising: (a) a first amino acid sequence comprising at least 35 or 100 amino acids, wherein the first amino acid sequence and the amino acid sequence of SEQ ID NO:8 have at least 85%, 90%, or 95% identity based on the Clustal alignment method, (b) a second amino acid sequence comprising at least 50 or 100 amino acids, wherein the second amino acid sequence and the amino acid sequence of SEQ ID NO:24 have at least 70%, 80%, 85%, 90%, or 95% identity based on the Clustal alignment method, (c) a third amino acid sequence comprising at least 100 amino acids, wherein the third amino acid sequence and the amino acid sequence of SEQ ID NO:2, SEQ ID NO:4, SEQ ID NO:20, or SEQ ID NO:22 have at least 80%, 85%, 90%, or 95% identity based on the Clustal alignment method, (d) a fourth amino acid sequence comprising at least 140 amino acids, wherein the fourth amino acid sequence and the amino acid sequence of SEQ ID NO:14 have at least 80%, 85%, 90%, or 95% identity based on the Clustal alignment method, (e) a fifth amino acid sequence comprising at least 150 amino acids, wherein the fifth amino acid sequence and the amino acid sequence of SEQ ID NO:10 or SEQ ID NO:12 have at least 80%,85%, 90%, or 95% identity based on the Clustal alignment method, (f) a sixth amino acid sequence comprising at least 200 amino acids, wherein the sixth amino acid sequence and the amino acid sequence of SEQ ID NO:6 have at least 80%, 85%, 90%, or 95% identity based on the Clustal alignment method, (g) a seventh amino acid sequence comprising at least 200 amino acids, wherein the seventh amino acid sequence and the amino acid sequence of SEQ ID NO:18 have at least 85%, 90%, or 95% identity based on the Clustal alignment method, or (h) an eighth amino acid sequence comprising at least 250 amino acids, wherein the eighth amino acid sequence and the amino acid sequence of SEQ ID NO:16 have at least 80%, 85%, 90%, or 95% identity based on the Clustal alignment method. The first amino acid sequence preferably comprises the amino acid sequence of SEQ ID NO:8, the second amino acid sequence preferably comprises the amino acid sequence of SEQ ID NO:24, the third amino acid sequence preferably comprises the amino acid sequence of SEQ ID NO:2, SEQ ID NO:4, SEQ ID NO:20, or SEQ ID NO:22, the fourth amino acid sequence preferably comprises the amino acid sequence of SEQ ID NO:14, the fifth amino acid sequence preferably comprises the amino acid sequence of SEQ ID NO:10 or SEQ ID NO:12, the sixth amino acid sequence preferably comprises the amino acid sequence of SEQ ID NO:6, the seventh amino acid sequence preferably comprises the amino acid sequence of SEQ ID NO:18, and the eighth amino acid sequence preferably comprises the amino acid sequence of SEQ ID NO:16. The polypeptide preferably is an acetolactate synthase small subunit.

The instant polypeptides (or portions thereof) may be produced in heterologous host cells, particularly in the cells of microbial hosts, and can be used to prepare antibodies to these proteins by methods well known to those skilled in the art. The antibodies are useful for detecting the polypeptides of the instant invention in situ in cells or in vitro in cell extracts. Preferred heterologous host cells for production of the instant polypeptides are microbial hosts. Microbial expression systems and expression vectors containing regulatory sequences that direct high level expression of foreign proteins are well known to those skilled in the art. Any of these could be used to construct a chimeric gene for production of the instant polypeptides. This chimeric gene could then be introduced into appropriate microorganisms via transformation to provide high level expression of the encoded acetolactate synthase small subunit. An example of a vector for high level expression of the instant polypeptides in a bacterial host is provided (Example 14).

Additionally, the instant polypeptides can be used as a target to facilitate design and/or identification of inhibitors of those enzymes that may be useful as herbicides. This is desirable because the polypeptides described herein catalyze the first committed step in branched chain amino acid biosynthesis. Accordingly, inhibition of the activity of one or more of the enzymes described herein could lead to inhibition of plant growth. ALS is also known to be the site of action of several diverse classes of herbicides including the sulfonylureas (Chaleff and Mauvais (1984) Science 224:1443-1445; LaRossa and Schloss (1984) J Biol Chem 259:8753-8757; Ray (1984) Plant Physiol 75:827-831). Thus, the instant polypeptides could be appropriate for new herbicide discovery and design.

All or a substantial portion of the polynucleotides of the instant invention may also be used as probes for genetically and physically mapping the genes that they are a part of, and used as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et al. (1987) Genomics 1:1-74-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4:37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel et al. In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask (1991) Trends Genet. 7:149-154). Although current methods of FISH mapping favor use of large clones (several to several hundred KB; see Laan et al. (1995) Genome Res. 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer (1989) Proc. Natl. Acad. Sci USA 86:9402-9406; Koes et al. (1995) Proc. Natl. Acad. Sci USA 92:8149-8153; Bensen et al. (1995) Plant Cell 7:75-84). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the instant polypeptide. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding the instant polypeptide can be identified and obtained. This mutant plant can then be used to determine or confirm the natural function of the instant polypeptides disclosed herein.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Isolation of the cDNA for the Small Subunit of Acetolactate Synthase

Embryos were harvested from ears of field grown LH195 corn 15 days post pollination and quickly frozen in liquid nitrogen. Total RNA was prepared from frozen tissue using guanidinium thiocyanate extraction and CsCl purification as described by Colbert et al. [Proc. Natl. Acad. Sci. USA 45 1703-1708 (1983)]. Total RNA was sent to Stratagene Cloning Systems (La Jolla, Calif.) where a directional cDNA library was made in the vector lambda Uni-ZAP™ using their custom cDNA library service. The library was plated at low density and individual plaques were randomly selected and picked into SM (50 mM Tris-HCl pH 7.5, 10 mM MgSO₄, 7H₂O, 10 mM NaCl, 0.01% gelatin). After elution of the phage from the agar plug, the resulting phage stocks were diluted 1:10, and cDNA insert was amplified from the phage using the polymerase chain reaction (PCR) using vector-directed primers (T3/T7). PCR products were purified using Qiagen PCR purification kit and an aliquot of the purified PCR product was checked by agarose gel electrophoresis for DNA integrity. The DNA was then sequenced using an Applied Biosystems Inc. (Foster City, Calif.) Model 373 DNA sequencer and ABI dye terminator sequencing kit. The resulting single pass cDNA sequence was compared to sequences in GenBank using NCBI Blast facility, and in this way, a clone designated m15.12.b12.sk20 was identified by the sequence similarity of its coding region to various bacterial small subunit of acetolactate synthase (SEQ ID NO:1).

Nicotiana plumbaginifolia was grown to the 5-leaf stage in Metromix 350 at 26° C. in a growth chamber maintained at 75% relative humidity using a 16 hr/8 hr day/night cycle. Plants were removed from soil with roots, rinsed in 0.5×Hoagland's solution to remove as much adhering soil as possible and frozen in liquid nitrogen. Total RNA was prepared from frozen tissue using guanidinium thiocyanate extraction and CsCl purification as described by Colbert et al. [Proc. Natl. Acad. Sci. USA 45 1703-1708 (1983)]. Total RNA was sent to Clontech Laboratories. Inc. (Palo Alto, Calif.) where a directional cDNA library in the vector lambda ZAP II™ was made using their custom cDNA library service.

A total of 3×10⁶ phage were plated on 25×25 cm square NZY plates (10 g NZ amine, 5 g yeast extract, 2 g MgSO₄) at a density of 2.5×10⁵ plaques/plate. To do this, 2.5×10⁵ phage were mixed with 5 mL of an overnight culture of E. coli XL-1 Blue grown in NZY containing 0.2% maltose and cells were incubated at 37° C. for 15 min. The infected culture was mixed with 40 mL of NZY top agar (ZY media containing 0.7% agarose) and pored onto the plate. After the top agarose hardened, plates were incubated at 37° C. for 6-8 h and then stored at 4° C. Phage lifts were then performed by layering dry MagnaGraph™ nylon transfer membranes (Micron Separations Inc., Westborough, Mass.) on top of the phage plates for 5 min. Membranes were transferred with their DNA side facing upward onto Whatman 3MM paper saturated with 0.5 M NaOH, 1.5 M NaCl. After 5 min, membranes were transferred onto Whatman 3MM paper saturated with 0.5 M Tris-HCl pH 7.5, 1.5 M NaCl and incubated for an additional 5 min. Membranes were then rinsed with 2×SSPE (20×SSPE is 3MM NaCl, 0.1 M Na₂HPO₄, 20 mM EDTA, pH 7.4), air dried for 10 min and DNA was crosslinked to the membrane using a Stratalinker™ UV Crosslinker (Stratagene; La Jolla, Calif.) in the “Auto Cross Link” mode by following the manufacturer's protocol. Membranes were stored at 4° C. in sealed polyethylene bags.

The cDNA insert from 25 μg of the m15.12.b12.sk20 plasmid was digested with Eco RI and Xho I and the digestion products were separated by electrophoresis using a 7.5% polyacrylamide gel. The gel was stained with ethidium bromide and the 750 bp cDNA fragment was recovered as described [Methods in Enzymology, Vol. 65 499-560, Academic Press, New York (1980)]. The DNA was labeled with ³²P by random priming using a RadPrime™ Labeling System kit (GibcoBRL, Gaithersburg, Md.) to a specific activity of >10⁹ dpm/μg as per the manufacturer's protocol. Following removal of unincorporated label by two precipitations of the DNA with ethanol, the labeled DNA was denatured by boiling for 5 min and quench cooling to 0° C. on ice.

Phage lift membranes were prehybridized for 4 h at 65° C. in 6×SSPE, 0.5% SDS, 1 mM EDTA, 5×Denhardt's solution, 100 μg denatured and sonicated calf thymus DNA/mL. Membranes were then hybridized overnight with the m15.12.b12.sk20 probe in 6×SSPE, 0.5% SDS, 1 mM EDTA, 2×Denhardt's solution, 100 μg denatured and sonicated calf thymus DNA/mL. The membranes were washed twice at room temperature with 2×SSPE, 0.1% SDS, twice with 2×SSPE, 0.1% SDS at 55° C., air dried and exposed to Kodak X-OMAT XAR-5 film overnight at −80° C. using a single intensifying screen. Agarose plugs containing plaques that hybridize to the probe were picked from appropriate plates and phage were eluted into 1 mL of SM (50 mM Tris-HCl pH 7.5, 0.1 M NaCl, 10 mM MgSO₄, 0.01% gelatin) by overnight incubation at 4° C. Plaque purification of phage was performed by serially diluting eluted phage with SM, infecting 100 uL cultures of E. coli XL-1 Blue with 100 uL aliquots of the dilutions and growing the infected bacteria overnight at 37° C. on NZY plates. Lifts of these plates were prepared and hybridized with labeled insert cDNA from the m15.12.b12.sk20 plasmid as described above. Hybridizing plaques were repeatedly subjected to this procedure until all plaques on a given plate hybridized with the probe DNA. In this manner, 12 phage isolates were prepared and stored at 4° C. in 0.5 mL of SM containing 50 uL of CHCl₃.

Plasmid DNA was obtained from each pure phage isolate as follows. Overnight cultures of E. coli XL-1 Blue and E. coli SOLR were grown up in NZY and LB media, respectively. XL-1 Blue cells were diluted to an A₆₀₀ of 1.0 with NZY and 200 uL of this dilution was infected with 100 uL of pure phage stock and 1 uL of a 10¹⁰ pfu/mL stock of ExAssist™ helper phage (Stratagene). Following a 15 min incubation at 37° C., cells were diluted with 3 mL of 2×YT media and incubated with shaking at 37° C. for 2 hr. The culture was heated to 70° C. for 20 min and then centrifuged at 10,000×g for 5 min at 4° C. The supernatant was transferred to a fresh tube and 2 uL of this supernatant was mixed with 200 uL of SOLR cells from the overnight culture. Following a 15 min incubation at 37° C., 10 and 100 uL aliquots of the cultures were spread onto LB plates supplemented with 100 μg ampicillin/mL and the plates were incubated at 37° C. overnight. Plasmids from individual antibiotic resistant colonies were analyzed for cDNA inserts by digestion with Eco RI and Xho I.

Sequence analyses were performed on plasmids harboring the largest sized inserts by the dideoxy sequencing method using a Sequenase™ DNA Sequencing Kit (Stratagene, La Jolla, Calif.). The 5′ end of the ALS small subunit mRNA was confirmed by rapid amplification of cDNA ends (5′ RACE) using a 5′/3′ RACE kit form Boehringer Mannheim (Indianapolis, Ind.). To this end total RNA prepared as described above was used as the target for RACE following the manufacturer's recommendation using oligodeoxynucleotide SU5R of the sequence

SU5R 5′-CCCAGTACGAGCAATTTCTC-3′ (SEQ ID NO:26)

to prime first strand cDNA synthesis. After dA tailing of the ss cDNA, 35 cycles of PCR was performed using the oligo dT anchor primer supplied with the 5′/3′ RACE kit and the specific ALS SSU primer SU4R

SU4R 5′-GTGGCTCCTTGGATAGATCT-3′ (SEQ ID NO:27)

using a temperature profile of: 95° C. for 1 min, 54° C. for 1 min, 72° C. for 1 min. The RACE products were separated by electrophoresis in a 2% agarose gel. A region of the gel containing 400-500 bp DNA fragments was excised and the DNA is purified using a QIAquick™ gel extraction kit (Qiagen, Inc., Chatsworth, Calif.). The purified RACE product was ligated into the vector pGEM-T (Promega Corp., Madison, Wis.) using conditions described earlier, 1 uL of the ligation reaction was used to transform competent E. coli JM109. Aliquots of the transformation mixture were spread on LB plates containing 100 μg ampicillin/mL and the plates were incubated at 37° C. overnight. Plasmids from individual antibiotic resistant colonies were analyzed for cDNA inserts by digestion with Nar I and Nco I. The inserts of clones showing the largest inserts were subjected to DNA sequence analysis as described above and the resulting RACE sequence was combined with that obtained from clones isolated from screening of a lambda ZAP II™ cDNA library to assemble the full-length sequence of the N. plumbaginifolia small subunit of acetolactate synthase cDNA. This clone was renamed pSSU.NP1, and is comprised of the sequence presented as SEQ ID NO:23 contained within the plasmid pBluescript(SK−).

### Example 2

### Construction of Acetolactate Synthase Small Subunit Expression Vectors Construction of pTrx-Bst1107

Five micrograms of oligodeoxynucleotides pTrx linker1 and pTrx linker2 were phosphorylated separately for 30 min at 37° C. in 100 uL of 50 mM Tris-HCl, pH 7.5, 10 mM MgCl₂, 10 mM dithiothreitol, 1 mM ATP and 100 units of T₄ polynucleotide kinase. 3.2 uL aliquots of each phosphorylated primer were then combined, diluted to 50 uL with H₂O and heated to 70° C. for 20 min and allowed to cool to ambient temperature.

pTrx linker1 5′-GTATACGAGGATCCTCTAGAG-3′ (SEQ ID NO:28)

pTrx linker2 5′-TCGACTCTAGAGAGTCCTCGTATAC-3′ (SEQ ID NO:29)

The bacterial expression vector pTrxFUS (Invitrogen) was digested to completion with Kpn I and the resulting 3′ overhang was removed by incubation of the DNA with the Klenow fragment of DNA polymerase I for 1 h at 25° C. in a buffer consisting of 50 mM Tris-HCl, 10 mM MgCl₂, 0.1 mM each of dATP, dCTP, dGTP, and dTTP, and 1 unit of Klenow/μg of DNA. The DNA was then extracted sequentially with equal volumes of phenol:CHCl₃:isoamyl alcohol (25:24:1) and CHCl₃ and precipitated on dry ice for 20 min after adding 0.1 volume of 3 M sodium acetate pH 6 and 2 volumes of ethanol. DNA was recovered by centrifugation at 14,000×g for 10 min. The DNA was digested to completion with Sal I and dephosphorylated with calf intestinal alkaline phosphatase. One μg of this DNA was then ligated with 1 uL of the dilute mixture of phosphorylated pTrx linker1 and pTrx linker2 prepared above at 16° C. for 3 h in 10 uL of 66 mM Tris-HCl, pH 7.6, 5 mM MgCl₂, 1 mM dithioerythritol, and 5 units of T₄ DNA ligase. The ligation reaction was diluted to 50 uL with H₂O and 2 uL of this dilution was used to transform competent Max Efficiency E. coli HB101 (GibcoBRL, Gaithersburg, Md.) using the protocol supplied with the cells. Aliquots of the transformation mixture were spread on LB plates containing 100 μg/mL ampicillin and the-plates were incubated at 37° C. overnight. Plasmids from individual antibiotic resistant colonies were analyzed for incorporation of the desired double-stranded oligonucleotide by dideoxy sequence analysis using the commercially available forward and reverse sequencing primers (Invitrogen). One clone found to contain the correct insert is designated pTrx-Bst1107.

Construction of pTrx-HIS

A polymerase chain reaction is performed using plasmid pTrxFUS as a target and the oligodeoxynucleotides HIS-TAG5 and HIS-TAG3 as primers.

HIS-TAG5 5′-GGAATTCTCCATATGCACCATCATCATCATCATAGCGATAAAAT TATTCAC-3′ (SEQ ID NO:30)

HIS-TAG3 5′-CCTGTACGATTACTGCAGGTC-3′ (SEQ ID NO:31)

The reaction mixture was assembled in a total volume of 500 uL containing 10 mM Tris-HCl, pH 8.3, 50 mM KCl, 1.5 mM MgCl₂, 0.001% gelatin, 0.2 mM each of dATP, dCTP, dGTP, dTTP, 800 ng plasmid pTrxFUS, 150 pmoles each of HIS-TAG5 and HIS-TAG3, and 12.5 units of AmpliTaq polymerase (Perkin Elmer Cetus, Norwalk, Conn.). The mixture was divided into 5 100 μL aliquots and the five tubes were subjected to 18 cycles of a temperature profile of: 95° C. for 1 min, 55° C. for 1 min, 72° C. for 1 min. A final cycle of 95° C. for 1 min, 55° C. for 1 min, 72° C. for 5 min completed the reaction. The five aliquots were pooled and extracted sequentially with equal volumes of phenol:CHCl₃:isoamyl alcohol (1:1:1) and CHCl₃. The DNA was precipitated by addition of 0.1 volume of 3M Msodium acetate pH 6 and 2 volumes of ethanol followed by a 20 min incubation on dry ice. DNA was recovered by centrifugation at 14,000×g for 10 min, dried in vacuo and dissolved in 100 uL of 10 mM Tris-HCl pH 7.5, 1 mM EDTA (TE).

One half of the PCR product was digested to completion with Nde I and Rsr II. The digest was extracted sequentially with equal volumes of phenol:CHCl₃:isoamyl alcohol (25:24:1) and CHCl₃. The DNA was precipitated by addition of 0.1 volume of 3 M sodium acetate pH 6 and 2 volumes of ethanol followed by 20 min on dry ice. DNA was recovered by centrifugation at 14,000×g for 10 min and the pellet was dried in vacuo and dissolved in 15 uL of TE. A 120 ng aliquot of Nde I/Rsr II digested PCR product was ligated with 1 μg of the vector pTrx-Bst1107 that had been digested to completion with Nde I and Rsr II and dephosphorylated with calf intestinal alkaline phosphatase in a 10 uL ligation reaction using conditions described above.

Competent E. coli GI 724 cells were prepared by following the protocol supplied by Invitrogen. A 4 uL aliquot of the ligation mixture was used to transform 110 uL of competent E. coli GI 724 cells using the chemical transformation protocol supplied by Invitrogen, Inc. Aliquots of the transformation mixture were spread on RMG plates [6 g/L Na₂HPO₄, 3 g/L K₂HPO₄, 0.5 g/L NaCl, 1 g/L NH₄Cl, 2% amicase (acid casein hydrolysate; Sigma Chemical Co., St. Louis, Mo.), 1 mM MgCl₂, pH 7.4, 0.5% glucose, 12.5% agar] containing 100 μg/mL ampicillin and the plates were incubated at 30° C. overnight. Plasmids from individual antibiotic resistant colonies were analyzed for inserts by dideoxy sequencing until a colony is found to containing the expected PCR product encoding a (His)₆ incorporated into the Nde I/Rsr II site of pTrx-Bst1107. This plasmid was designated pTrx-HIS.

Construction of pTrx-HIS/SSU

A polymerase chain reaction was performed using cDNA clone SSU.NP1 as a target and the oligodeoxynucleotides SSU-PCR1 and SSU-PCR3 as primers.

SSU-PCR1 5′-AACAACAACGATATCAGACAAAAGACTAGGCGCCA-3′ (SEQ ID NO:32)

SSU-PCR3 5′-AACAACGGATCCAACCAACTTATATAGTTGCTGCACCA-3′ (SEQ ID NO:33)

PCR was performed using the conditions described above with 150 pmoles each of SSU-PCR1 and SSU-PCR3, and 800 ng of SSU.NP1 in the reaction. The PCR reaction was then extracted, precipitated, and redissolved in 100 uL of TE as described earlier.

One half of the PCR product was digested to completion with Eco RV and Bam HI. The digest was then extracted sequentially with equal volumes of phenol:CHCl₃:isoamyl alcohol (1:1:1) and CHCl₃. The DNA was precipitated by addition of 0.1 volume of 3M Msodium acetate pH 6 and 2 volumes of ethanol followed by 20 min on dry ice. DNA was recovered by centrifugation at 14,000×g for 10 min. The pellet was dried in vacuo and dissolved in 15 uL of TE. Approximately 250 ng of Eco RV/Bam HI digested PCR product was ligated with 1 μg of the vector pTrx-HIS that had been digested to completion with Bst11071 and Bam HI and dephosphorylated with calf intestinal alkaline phosphatase in a volume of 10 uL. A 4 uL aliquot of the ligation mixture was used to transform competent E. coli G1724 cells as described above and aliquots of the transformation mixture were spread on RMG plates and incubated at 30° C. overnight. Plasmids from individual antibiotic resistant colonies were analyzed for inserts by digestion with Nde I and Xba I until a colony was found that contains a plasmid showing a 1.7 kbp Nde I/Xba I insert. This plasmid was designated pTrx-HIS/SSU.

The same Eco RV/Bam HI digested PCR product was ligated with 1 μg of pTrx-Bst1107 that had been previously digested with Bst1107 and BamHI to yield the plasmid pTrx-BST/SSU.

Construction of pGEX-2TM

Additional restriction sites were added to the multiple cloning site of protein expression vector pGEX-2T (Pharmacia Biotech, Uppsala, Sweden). pGEX-2T (10 μg) was digested to completion with BamH I and EcoRI and the digestion products were separated by electrophoresis using a 1% agarose gel. The 4.95 kbp band was excised from the gel and the DNA was recovered using a QIAquick™ Gel Extraction Kit (Qiagen, Chatsworth, Calif.) according to the manufacturer's instructions. Sodium acetate (pH 5.2) was added to the final column eluant to give a concentration of 0.3 M; 10 μg of tRNA and 2 volumes of cold ethanol were then added and the DNA was recovered by centrifugation. The pellet was washed with 70% ethanol, air dried at room temp, dissolved in 10 μL TE and quantified by running a 1 μL aliquot on a 1% agarose gel and comparing the band intensity with that of a commercial mass ladder standard.

Approximately 50 ng of digested plasmid were mixed with 70 ng each of phosphorylated CAM19 and CAM20 oligonucleotides and the mixture was incubated at 45° C. for 5 minutes and cooled on ice. The ligated oligonucleotides create the following sites: BamH I, NcoI, SalI, XhoI, PinAI and EcoRI.

CAM19 5′-GATCCATGGTCGACTCGAGACCGGTG-3′ (SEQ ID NO:34)

CAM20 5′-AATTCACCGGTCTCGAGTCGACCATG-3′ (SEQ ID NO:35)

T4 DNA ligase buffer and 1 unit of T4 DNA ligase (GibcoBRL) were added and the mixture was incubated at 37° C. for 1 hour. The ligation mix was then transformed into DH5α Max Efficiency Competent E. coli (GibcoBRL) using a standard heat shock protocol (Sambrook) and spread onto LB plates containing 100 μg carbenicillin/mL. The plates were incubated overnight at 37° C. and plasmids were isolated from carbenicillin-resistant bacterial colonies and analyzed for the presence of both PinAI and NcoI restriction sites by restriction endonuclease digestion. A plasmid with PinAI and NcoI sites was selected and designated pGEX-2TM.

Construction of pGEX-SSU

Five micrograms of oligodeoxynucleotides SSU oligo 9 and SSU oligo 10 were phosphorylated separately for 30 min at 37° C. in 100 μL of 50 mM Tris-HCl, pH 7.5, 10 mM MgCl₂, 10 mM dithiothreitol, 1 mM ATP and 100 units of T₄ polynucleotide kinase.

SSU oligo 9 5′-GATCTGAAGACAACATGATCAGACAAAAGACTAGGCGCC AACAACAAGGATCC-3′ (SEQ ID NO:36)

SSU oligo 10 5′-TCGAGGATCCTTGTTGTTGGCGCCTAGTCTTTTGTCTGA TCATGTTGTCTTCA-3′ (SEQ ID NO:37)

Four μL aliquots of each phosphorylated primer were combined, diluted to 40 μL with H₂O, heated to 70° C. for 20 min and allowed to cool to ambient temperature.

The bacterial plasmid vector pBluescript SK(+) (Stratagene, La Jolla, Calif.) was digested to completion with Bam HI and Xho I dephosphorylated with calf intestinal alkaline phosphatase. One μg of this DNA was then ligated with 1 μL of the mixture of phosphorylated SSU oligo 9 and SSU oligo 10 at 16° C. for 3 h in 10 μL of 66 mM Tris-HCl, pH 7.6, 5 mM MgCl₂, 1 mM dithioerythritol using 5 units of T₄ DNA ligase. The ligation reaction was diluted to 50 μL with H₂O and 2 μL was used to transform competent Max Efficiency E. coli HB101 (Gibco/BRL, Gaithersburg, Md.) using the protocol supplied with the cells. Aliquots of the transformation mixture were spread on LB plates containing 100 μg/mL ampicillin and the plates were incubated at 37° C. overnight. Plasmids from individual antibiotic resistant colonies were analyzed for incorporation of the desired double-stranded oligonucleotide by dideoxy sequence analysis using the commercially available forward and reverse sequencing primers. One clone found to contain the correct insert was designated pSSU 9/10.

Five micrograms of oligodeoxynucleotides SSU oligo 5 and SSU oligo 6 were phosphorylated as described above.

SSU oligo 5 5′-TGCGGAATGTGTGCGAACGTGGATGACTGAATGGA TCCGGTAC-3′ (SEQ ID NO:38)

SSU oligo 6 5′-CGGATCCATTCAGTCATCCACGTTCGCACACATTC-3′ (SEQ ID NO:39)

Two μL aliquots of each phosphorylated primer were then combined, diluted to 40 μL with H₂O, heated to 70° C. for 20 min and allowed to cool to ambient temperature.

The bacterial plasmid SSU.NP1 was digested to completion with Kpn I and BspMI and dephosphorylated with calf intestinal alkaline phosphatase. One mg of this DNA was then ligated with 1 μL of the dilute mixture of phosphorylated SSU oligo 5 and SSU oligo 6 as described above. The ligation reaction was diluted to 50 μL with H₂O and used to transform competent Max Efficiency E. coli HB101 as described above. Plasmids from individual antibiotic resistant colonies were analyzed for incorporation of the desired double-stranded oligonucleotide by digestion with Bam HI. One clone found to contain the correct 1.33 kbp Bam HI insert was designated pNP1 5/6.

The plasmid pNP1 5/6.was digested to completion with Kas I and Bam HI and the digestion products were separated by electrophoresis using a 6.5% agarose gel. The 1.3 kbp Kas I/Bam HI fragment was recovered from the gel. 400 ng of this DNA was ligated with 1 μg of the plasmid pSSU 9/10 that had been digested to completion with Kas I and Bam HI and dephosphorylated as described above. The ligation reaction was diluted five fold with H₂O and used to transform competent Max Efficiency E. coli HB101 as described above. Plasmids from individual antibiotic resistant colonies were analyzed by digestion with Bbs I and Xho I until a clone found was found that contained the desired 1.3 kbp insert. This plasmid was designated pSSUBbs/Bam.

The plasmid pSSU Bbs/Bam was digested to completion with Bbs I and Xho I and the digestion products were separated by electrophoresis using a 6.5% agarose gel and the 1.35 kbp Bbs I/Xho I fragment was recovered from the gel. Three hundred ng of this DNA was ligated with 1 μg of the plasmid pGEX-2TM that had been digested to completion with BamH I and Xho I and dephosphorylated as described above. The ligation reaction was diluted five fold with H₂O and used to transform competent Max Efficiency E. coli DH5α as described above. Plasmids from individual antibiotic resistant colonies were analyzed by restriction endonuclease digestion until a clone found was found that contained the desired ALS small subunit coding region. This plasmid was designated pGEX-SSU.

### Example 3

### Expression of Recombinant Plant Acetolactate Synthase Small Subunit Expression of Trx-HIS/SSU

The strain G1724 harboring the plasmid pTrx-HIS/SSU (designated G1724/Trx-HIS/SSU) was struck onto a fresh RMG plate containing 100 μg/mL ampicillin and grown overnight at 30° C. The next day, a single colony was then used to inoculate a 20 mL culture of RM (6 g/L Na₂HPO₄, 3 g/L K₂HPO₄, 0.5 g/L NaCl, 1 g/L NH₄Cl, 20 g/L amicase (acid casein hydrolysate; Sigma Chemical Co., St. Louis, Mo.), 1 mM MgCl₂, pH 7.4, 1% glycerol) supplemented with ampicillin to 100 μg/mL was grown overnight at 30° C. On the third day, the A₅₅₀ of the culture was measured and 1 L of induction media [6 g/L Na₂HPO₄, 3 g/L K₂HPO₄, 0.5 g/L NaCl, 1 g/L NH₄Cl, 0.2% amicase (acid casein hydrolysate; Sigma Chemical Co., St. Louis, Mo.), 1 mM MgCl₂, pH 7.4, 0.5% glucose, 100 μg/mL ampicillin] was inoculated with a sufficient volume of the overnight culture to give an initial A₅₅₀ of 0.1. This fresh culture was grown to an A₅₅₀ of 0.6 and 10 mL of a 10 mg/mL solution of tryptophan was added. The culture was incubated at 28° C. for 3-4 h and then chilled to 0° C. in a ice bath and subjected to centrifugation at 8000×g for 10 min. The supernatant was discarded and the cell pellet was frozen at −80° C.

Expression of GST-SSU

A single colony of BL21 transformed with pGEX-SSU from an LB/Amp (100 μg/mL) plate was used to inoculate 10 mL of LB containing 100 μg/mL ampicillin. These cultures were allowed to incubate overnight with shaking at 28° C. Two milliliters of the overnight culture was used inoculate each liter of LB containing 100 μg/mL ampicillin. The cultures were grown at 28° C. until the absorbency at 600 nm was equal to 0.6. At this stage, 1 mL of 0.1 g/mL IPTG was added to each culture. The cultures were allowed to remain at 28° C. for 5 h with shaking after which time the cultures were placed on ice and the cells were harvested by centrifugation at 8000 rpm in a GS3 rotor for 15 min. The cells were stored frozen at −80° C. until use.

### Example 4

### Purification of Plant ALS Small Subunit

Purification of Trx-HIS/SSU

The small subunit of plant ALS can be purified, using the appropriate affinity resin, as a fusion protein with a variety of affinity tags including but not exclusive to thioredoxin, hexahistidine and glutathione-S-transferase. The following example is for purification of the small subunit as a fusion protein with thioredoxin. Approximately 11 g of G1724 cells harboring the plasmid pTrx-HIS/SSU were suspended in 33 mL of buffer containing 5 mM imidazole, 500 mM NaCl, 20 mM Tris-HCl pH 7.9 and 0.5% Triton X-100. The resuspended cells were sonicated using a half-inch horn on a Heat Systems Sonicator. Cells were sonicated, on ice, at full power using a 50% pulsed duty cycle for 15 s followed by 45 s rest. This sequence was repeated 19 times. The cell lysate was centrifuged at 10,000 rpm for 15 min in an SS34 rotor. The supernatant was transferred to a clean centrifuge tube and the centrifugation was repeated until the supernatant was clear. The lysate supernatant was added to 20 mL of ThioBond™ resin (Invitrogen). Binding of the fusion protein was allowed to proceed for 45 min by gently rocking the resin at room temperature. The resin was allowed to settle and the liquid was decanted. Unbound protein was removed by washing the resin with 40 mL of buffer containing 50 mM MOPS, pH 7.0 and 1 mM EDTA. The resin was equilibrated with this buffer by gentle rocking and then allowed to settle and the supernatant was removed. This procedure was repeated twice. The fusion protein was eluted by washing the resin with buffer containing 50 mM MOPS pH 7.0, 1 mM EDTA, 10 mM DTT and 10% glycerol. After equilibration, the supernatant was decanted and the procedure was repeated. The fusion protein was eluted with a total of 100 mL of buffer. The eluted protein was concentrated using an Amicon concentrator and a PM30 membrane. The sample was dialyzed into PBS buffer containing 10% glycerol and then frozen at −80° C for storage.

Purification of GST-SSU

Approximately 50 g of BL21 cells harboring the plasmid pGEX-SSU were suspended in 100 mL of 1×PBS buffer. The cell suspension was subjected to three passes through a microfluidizer. A 10% Triton X-100 solution and a 20% sarkosyl solution were each added to the cell lysate to a final concentration of 1%. The solution was allowed to equilibrate for 30 min at room temperature with gentle agitation. The cell debris was then centrifuged at 10,000 rpm in a GSA rotor for 30 min. The supernatant was removed and added to 100 mL of glutathione agarose (Sigma). The resin and lysate were allowed to equilibrate at room temperature for 30 minutes with gentle rocking. The supernatant was removed by centrifugation at 500×g (swinging bucket rotor) for 5 minutes. The supernatant was decanted and the resin was washed with 200 mL of 1×PBS. The resin and PBS were allowed to equilibrate at room temperature for 10 minutes and with rocking and the supernatant was removed by centrifugation as above. The washing procedure was repeated a total of 4 times. The bound GST-SSU was eluted by adding 200 mL of 25 mM Ches pH 9 containing 10 mM reduced glutathione. The resin was equilibrated with the Ches buffer for 10 min with gentle rocking at room temperature. The supernatant was collected by centrifugation as above. The elution procedure was repeated a total of three times. The eluted protein was concentrated to 9 mL using a Millipore ultrafiltration apparatus. The protein was dialyzed extensively to remove the glutathione and then stored frozen at −80° C.

### Example 5

### Expression and Purification of an Arabidopsis thaliana ALS Large Subunit

The ALS large subunit may also be cloned into a variety of expression vectors and purified as a fusion protein using numerous affinity chromatography strategies specific for, but not exclusive to, thioredoxin, glutathione-S-transferase and hexahistidine. The procedure described below can be used for any ALS large subunit cloned into a pGEX expression vector [“GST Gene Fusion System” Pharmacia Biotech, 1996; Bernasconi et al., J. of Biol. Chem. 270: 17381-17385 (1995)].

The plasmid pGATX was created by cloning the 5.8 kbp Xba I fragment of the genomic lambda phage clone 7 [Mazur et al. Plant Physiol. 85:1110-111 (1987)], into the plasmid vector pGEM1 (Promega). This 5.8 kbp Xba I fragment contains a complete copy of the Arabidopsis thaliana ALS large subunit gene. The plasmids pGEX-2TM (see Example 2) and pGATX were both digested to completion with Nco I and the digests then placed on Qiagen QIAquick™ columns and the Nucleotide Removal Kit protocol followed. The final column eluant was adjusted to 1×React 4 (Gibco BRL) and the DNAs were digested to completion with PinAI. The pGEX-2TM digest was heated to 65° C. for 10 minutes to destroy remaining PinAI activity and the DNA was dephosphorylated by incubating it at 37° C. for 30 min. with 1 unit of calf intestinal alkaline phosphatase. Both pGEX-2TM and pGATX were subjected to electrophoresis using a 1% agarose gel. The 2 kbp fragment from pGATX and the 4.9 kbp fragment from pGEX-2TM were removed from the gel and the DNAs were recovered using a QIAquick™ Gel Extraction Kit according to the manufacturer's instructions. Purified DNAs were precipitated, re-dissolved and quantified as above. Approximately 90 ng of 2.0 kbp pGATX fragment were mixed with 120 ng of the NcoI/PinAI digested pGEX-2TM and mixture was heated for 5 minutes at 45° C. followed by cooling on ice. T4 DNA ligase buffer and 1 unit of T4 DNA ligase were mixed with the fragments and mixture was incubated for 4 hours at ambient temperature. An aliquot of the ligation mixture was used to transform competent E. coli DH5α as described above and aliquots of the transformation mixture were spread onto LB plates containing 100 μg/mL carbenicillin. The plates were incubated overnight at 37° C. and plasmids were isolated from carbenicillin-resistant bacterial colonies were analyzed for the presence of the ALS insert by double digestion with PinAI and NcoI. One plasmid thus isolated was designated pGEX-OCM2 was then transformed into E. coli. BL21(DE3) using methods known to those skilled in the art.

A single colony of BL21(DE3) transformed with pGEX-OCM2 (encoding a fusion protein designated GST-ALS, comprising the glutathione-S-transferase fused to the Arabidopsis thaliana large subunit of acetolactate synthase, including the chloroplast transit peptide) from an LB/Amp (100 μg/mL) plate is used to inoculate 10 mL of LB containing 100 μg/mL carbenicillin. These cultures were allowed to incubate overnight with shaking at 30° C. Two milliliters of the overnight culture was used inoculate each liter of LB containing 100 μg/mL carbenicillin. The cultures were grown at 30° C. until the absorbency at 600 nm was equal to 0.6. At this stage, 1 mL of 0.1 g/mL IPTG was added to each culture. The cultures were allowed to remain at 30° C. for 4 h with shaking after which time the cultures were placed on ice and the cells were harvested by centrifugation at 8000 rpm in a GS3 rotor for 15 min. The cells were stored frozen at −80° C. until use.

Approximately 11 grams of cells were resuspended in 20 mL of PBS buffer containing Complete™ protease inhibitors (Boehringer Mannheim, Indianapolis, Ind.). The cells were lysed by sonication (Heat Systems) 19×15 s with 45 s rest on ice using a microtip at power level 4 and 50% duty cycle. The appropriate amount of a 20% Triton X-100 solution was added so that the final concentration of Triton in the lysate was 1%. The lysate was gently rocked at 4° C. for 30 min. The cell debris was removed by centrifugation at 12,000 rpm for 15 min in a SS34 rotor. The supernatant was removed and the cell pellet was discarded. Half of the cell lysate supernatant was added to a 2 mL column of glutathione Sepharose 4B (Pharmacia Biotech) equilibrated with PBS. The eluant was collected for all washes and elutions. The column was washed with 20 mL of PBS. The bound fusion protein was eluted using 5 mM glutathione (reduced) in PBS at pH 8.0. One column volume of elution buffer was added to the column and allowed to run into the column. The bottom of the column was capped and the column was allowed to sit at room temperature for 10 min. The column effluent was collected as another 2.66 mL of elution buffer was added. The process was repeated 3 times. The elution fractions were combined and dialyzed against 50 mM Hepes, pH 7.0 containing 10% glycerol and 0.1 mM FAD for several hours and then concentrated to 9 mL using a Centriprep 10 (Amicon).

### Example 6

### Expression and Purification of Nicotiana plumbaginifolia ALS Large Subunit

Equimolar amounts of oligodeoxynucleotides mt704+ (SEQ ID NO:40) and mt800-(SEQ ID NO:41) were combined in 100 uL of 50 mM Tris-HCl, pH 7.5, 10 mM MgCl₂, heated briefly in a boiling water bath and slow cooled to 50° C. The resulting double stranded DNA fragment was digested to completion with Nde I and Eco0109I.

The plasmid ALS10 that contains a full length cDNA copy of the Nicotiana plumbaginifolia ALS gene in the plasmid vector pBluescript was used as the source of the ALS coding region for pMTDRALS. The sequence of this cDNA is shown in SEQ ID NO:42. ALS10 was digested to completion with Xho I and Eco0109I, and the digestion products were separated by agarose gel electrophoresis. The 1.9 kbp Xho I/Eco0109I ALS10 DNA fragment was excised from the gel and the DNA was recovered using a QIAquick™ Gel Extraction Kit (Qiagen, Chatsworth, Calif.) according to the manufacturer's instructions. Equimolar amounts of Nde I/Eco090I digested double-stranded oligonucleotide mt704+/mt800− DNA, and 1.9 kbp Xho I/Eco01091 ALS 10 DNA were ligated together overnight. DNA ligase was inactivated by heating the mixture to 65° C. for 1 hr and the ligation products were digested to completion with Nde I and Xho I. The digestion products were separated by agarose gel electrophoresis and the 2.0 kbp Xho I/Nde I fragment was excised from the gel and the DNA was recovered as described earlier. The plasmid pET24a (Novagen, Inc., Madison, Wis.) was digested to completion with Nde I and Xho I and the DNA was dephosphorylated with calf intestinal alkaline phosphatase. Equimolar amounts of Nde I/Xho I digested and dephosphorylated pET24a and the 2.0 kbp Xho I/Nde I digestion product were ligated together and an aliquot of the ligation mixture was used to transform competent E. coli DH5a as described above. Aliquots of the transformation mixture were spread onto LB plates containing 100 μg/ml kanamycin. The plates were incubated overnight at 37° C. and plasmids isolated from kanamycin-resistant bacterial colonies were analyzed for the presence of the insert encoding the ALS large subunit by double digestion with Nde I and Xho I. One such plasmid, designated pMTDRALS was then transformed into E. coli BL21(DE3) using methods known to those skilled in the art. This plasmid directs the expression of the Nicotiana plumbaginifolia ALS large subunit.

A single colony of BL21 transformed with pMTDRALS grown on an LB/kanaymycin (30 μg/mL) plate was used to inoculate 50 mL of LB/kanamycin. This culture was grown overnight at 37° C. with agitation. This overnight culture was in turn used to inoculate 1 liter of minimal M9 medium supplemented with 2 mL 1M MgSO₄, 1 mL CaCl₂, 50 μL 1% vitamin B1, 40 mL of 10% casamino acids and 30 μg/mL kanamycin. The cells were grown until OD₆₀₀ was equal to 0.6 and expression was induced by the addition of IPTG to a final concentration of 0.5 mM. The cells were harvested by centrifugation 5 hours later.

Cells were washed and then resuspended in two volumes of 20 mM Tris, pH 7.8 containing 1 mM EDTA, 1 mM β-mercaptoethanol and 0.1 mM PMSF. The suspension was subjected to three passes through a microfluidizer and the lysed cell debris removed by centrifugation at 18000 rpm on a SS34 rotor. The proteins in the supernatant were fractionated by ion exchange chromatography using a DE 52 column equilibrated with 50 mM MOPS, pH 6.85, 1 mM EDTA and 1 mM S-ME. Protein was eluted with a linear NaCl gradient between 0 and 0.5 M. The tubes containing ALS activity were combined and the protein precipitated with 50% ammonium sulfate. The precipitated protein was centrifuged at 11000 rpm (GSA rotor) and the pellet resuspended in Tris, pH 7.2 containing 1 mM EDTA, 1 mM DTT and 20 μM FAD. The solutions were kept dark. The redissolved protein was desalted and fractionated using an S300 filtration column equilibrated with the FAD containing Tris buffer. The tubes containing ALS activity were combined, concentrated and the material loaded onto a MonoQ column equilibrated with Tris, pH 7.2 containing 1 mM EDTA, 1 mM DTT and developed with a linear NaCl gradient to 0.5M. The resulting enzyme was concentrated and washed with Tris buffer and stored as frozen aliquots in 10% glycerol. The exposure of solutions to light was minimized during this purification.

### Example 7

### Effect of the Small Subunit on ALS-LSU Activity

As discussed above, one way to prepare the ALS holoenzyme is to mix purified or partially purified large subunit with purified or partially purified small subunit. A typical subunit mixing experiment which demonstrates the effect of the small subunit on the specific activity of the LSU consists of the samples and the results shown in Table 2.

TABLE 2

| | | | | | Specific Activity⁴ | |
|---|---|---|---|---|---|---|
| | GST- | LSU² | Trx-HIS/ | BSA | before | after |
| Sam- | LSU¹ | (0.7 | SSU³ | (2 mg/ | thrombin | thrombin |
| ple | (1 mg/mL) | mg/mL) | (2 mg/mL) | mL) | addition | addition |
| 1 | 20 μl | — | — | 10 μl | 0.3 | 0.04 |
| 2 | 20 μl | — | 10 μl | — | 0.9 | 1.1 |
| 3 | — | 5 μl | — | 10 μl | 1.1 | 0.40 |
| 4 | — | 5 μl | 10 μl | — | 5.5 | 6.5 |

¹A. thaliana LSU expressed as a glutathione-S-transferase fusion protein from plasmid pGEX-OCM2.
²N. plumbaginifolia LSU from plasmid pMTDRALS.
³N. plumbaginifolia SSU expressed as a thioredoxin fusion from plasmid pTrx-HIS/SSU.
⁴Expressed as units/mg LSU.

Each sample contained 3 μl of 10×PBS and distilled, deionized water was added to each sample to bring the final volume to 30 μL. When small subunit was not added to the sample, an equivalent amount of BSA was added as a control protein. The Trx-HIS/SSU used in these experiments contains a thrombin cleavage site. Thus, the addition of thrombin leads to cleavage of the fusion to yield free SSU and Trx-HIS. Two different large subunits are used in this example. One ALS-LSU (GST-LSU) is a fusion between GST and the LSU of Arabidopsis ALS which contains the complete chloroplast transit peptide on the N-terminus of the LSU. Upon the addition of thrombin, the GST-LSU fusion is cleaved to give free GST and Arabidopsis LSU. The other ALS-LSU (LSU) is from Nicotiana plumbaginifolia and contains only a partial chloroplast transit peptide on the N-terminus as described above and contains no thrombin cleavage site. Each sample was assayed for ALS activity (see below) prior to thrombin addition. After the initial activity had been measured, 1 μL of thrombin (Pharmacia BioTech, 1 unit/μL) was added to each sample and the samples were incubated at room temperature for 2 h. Thrombin cleavage was shown to be complete by SDS Page analysis and no proteolytic degradation of the Nicotiana LSU was observed. Each sample was again assayed for activity. The assay results are shown in the last two columns of Table 2 where specific activities are calculated based upon large subunit concentration and are reported in terms of units/mg LSU.

The results in Table 2 show that the presence of the small subunit increases the specific activity of the LSU before and after thrombin cleavage of the fusion protein regardless of whether the LSU is from Arabidopsis or Nicotiana and that the SSU positively affects the stability of the LSU activity. A comparison of the specific activity of sample 1 and 2 before thrombin addition shows that when Trx-HIS/SSU is added to GST-LSU from Arabidopsis there is a 3-fold increase in specific activity even with fusion proteins attached to both large and small subunits. A comparison of the specific activities of samples 3 and 4 prior to thrombin addition shows that the presence of the Trx-HIS/SSU increases the specific activity of the Nicotiana LSU by a factor of 5. After thrombin addition to sample 1, the specific activity of the Arabidopsis LSU, now minus the GST fusion, decreases from 0.3 to 0.04 units/mg LSU. However, in the presence of SSU and thrombin, the specific activity of the Arabidopsis LSU increases slightly compared to the same sample in the absence of thrombin (sample 2) to 1.1 units/mg LSU representing more than a 20 fold increase in specific activity compared to the Arabidopsis LSU alone. In sample 3, the addition of thrombin does not cleave the Nicotiana large subunit, but the enzyme loses some activity during the incubation period. Control samples identical to 3 but which do not contain thrombin showed a similar decrease in specific activity over the 2 hour incubation period (data not shown). However, in the presence of the SSU and thrombin, the specific activity of the Nicotiana LSU increases from 5.5 to 6.5 (sample 4). Considering the loss in specific activity observed for the Nicotiana LSU alone during the incubation time, the SSU serves to not only increase the specific activity of the LSU but also serves to stabilize the LSU. Samples identical to 1 and 3 but which do not contain BSA showed similar specific activities to those with BSA. Thus, the effect of the small subunit cannot be mimicked by interactions with a non-specific protein. These results show not only that the small subunit of plant ALS increases the specific activity of the large subunit but also that the SSU from N. plumbaginifolia can affect the activity of an Arabidopsis large subunit as well. Thus the small subunit from one species of plant may affect the activity of a large subunit from another species.

ALS assays were conducted using the following reaction mixture containing 100 mM sodium phosphate pH 7.6, 0.5 mM dithiothreitol, 1 mM MgCl₂, 100 μM thiamine pyrophosphate, and 100 μM flavin adenine dinucleotide. The assay was be conducted in a microtiter plate wherein each well contained 80 μL of assay mix and 10 μL of 500 mM sodium pyruvate. The mixture was allowed to equilibrate for 5 min at 37° C. The desired amount of enzyme to be assayed was then added to the well and mixed by gentle shaking. The plate was incubated for the desired reaction time at 37° C. The reaction was quenched by the addition of 10 μL 3M MH₂SO₄ to each well. The contents of the plate were mixed well with gentle shaking and incubated at 60° C. for 15 min. The amount of acetoin produced was detected by the rapid addition of 100 μL of 0.5% creatine and 100 μL of 5% α-naphthol in 2.5M MNaOH. The contents of the plate were mixed and the plate was incubated for 15 min at 60° C. uncovered. The plate was cooled to room temperature for 5 min with constant gentle shaking and the absorbency at 530 nm was read. The specific activity can be calculated based upon the concentration of the large subunit given that 1 μmole of acetoin produces an absorbency of 0.35 under these conditions.

### Example 8

A holoenzyme mixture was prepared containing 50 μL of N. plumbaginifolia LSU (0.7 mg/mL; encoded by pMTDRALS), 60 μL GST-SSU (1.22 mg/mL; encoded by pGEX-SSU)), 10 μL distilled, deionized water, and 180 μL of a buffer/cofactor mixture containing 100 mM phosphate, pH 7.6, 10% glycerol, 0.5 mM DTT, 2 mM MgCl₂, 200 μM FAD and 200 μM TPP. The fusion proteins were cleaved by the addition of 1 μL of Thrombin (0.8 units/mL; Novagen) to the mixture and the solution was allowed to incubate at 25° C. for several hours. A microtiter plate was prepared in which each well contains 80 μL of assay mix (see ALS assay procedure above) and 10 μL of a stock solution containing the desired concentration of the inhibitor to be tested. The plate was incubated at 37° C. for 5 min and then 1 μL of the holoenzyme mixture was added to each well. The reaction was started by the immediate addition of 10 μL of 500 mM sodium pyruvate to each well. The contents of the plate were mixed well by brief agitation and then the plate was incubated at 37° C. for 30 min prior to quenching with 3 M H₂SO₄. The acetoin produced was detected as described above under ALS assays. The results of such a study using the commercial herbicides imazequin (Scepter®) and imezethapyr (Pursuit®) are shown in Table 3.

TABLE 3

| Compound | I₅₀ (μM) |
|---|---|
| imazequin | 0.80 |
| imezethapyr | 4.0 |

### Example 9

### Composition of cDNA Libraries: Isolation and Sequencing of cDNA Clones

cDNA libraries representing mRNAs from various corn, rice, soybean, and wheat tissues were prepared. The characteristics of the libraries are described below.

TABLE 4
cDNA Libraries from Corn, Rice, Soybean, and Wheat

| Library | Tissue | Clone |
|---|---|---|
| cen3n | Corn Endosperm 20 Days After Pollination* | cen3n.pk0112.c11 |
| m15 | Corn 15 Day Old Embryo | m15.12.b12.sk20 |
| p0094 | Corn Leaf Collars for the Ear Leaf (EL), | p0094.csstl72ra |
| | and the Next Leaf Above and Below the EL; | |
| | Growth Conditions: Field; Control or | |
| | Untreated Tissues* | |
| rl0n | Rice 15 Day Old Leaf* | rl0n.pk084.a24 |
| | | rl0n.pk117.a16 |
| sdc2c | Soybean Developing Cotyledon (6-7 mM) | sdc2c.pk001.b10 |
| wdk2c | Wheat Developing Kernel, 7 Days After | wdk2c.pk015.a13 |
| | Anthesis | |

*These libraries were normalized essentially as described in U.S. Pat. No. 5,482,845, incorporated herein by reference.

cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred/Phrap (P. Green, University of Washington, Seattle). Phrep/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (D. Gordon, University of Washington, Seattle).

### Example 10

### Identification of cDNA Clones

cDNA clones encoding ALS small subunits were identified by conducting BLAST (Basic Local Alignment Search Tool; Altschul et al. (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 9 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis are compared to the genbank database as described above. ESTs that contain sequences more 5- or 3-prime can be found by using the BLASTn algorithm (Altschul et al (1997) Nucleic Acids Res. 25:3389-3402.) against the DuPont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described in Example 9. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 11

### Characterization of Additional cDNA Clones Encoding ALS Small Subunit

The BLASTX search using the EST sequences from clones listed in Table 5 revealed similarity of the polypeptides encoded by the cDNAs to ALS small subunit from Nicotiana plumbaginifolia (NCBI GenBank Identifier (GI) No. 5931761). Shown in Table 5 are the BLAST results for individual ESTs (“EST”), the sequences of the entire cDNA inserts comprising the indicated cDNA clones (“FIS”), the sequences of contigs assembled from two or more ESTs (“Contig”), sequences of contigs assembled from an FIS and one or more ESTs (“Contig*”), or sequences encoding an entire protein derived from an FIS, a contig, or an FIS and PCR (“CGS”):

TABLE 5
BLAST Results for Sequences Encoding Polypeptides Homologous
to ALS Small Subunit

| | | BLAST pLog Score |
|---|---|---|
| Clone | Status | NCBI GenBank Identifier (GI) No. 5931761 |
| m15.12.b12.sk20 | EST | 52.40 |
| cen3n.pk0112.c11 | EST | 51.70 |
| p0094.csstl72ra | EST | 18.30 |
| rl0n.pk084.a24 | EST | 51.70 |
| rl0n.pk117.a16 | EST | 36.00 |
| sdc2c.pk001.b10 | CGS | 172.00 |
| (FIS) | | |
| wdk2c.pk015.a13 | EST | 31.00 |

The sequence of the entire cDNA insert in several of the clones listed in Table 5 was determined. The BLASTX search using the EST sequences from clones listed in Table 6 revealed similarity of the polypeptides encoded by the cDNAs to ALS small subunit from Nicotiana plumbaginifolia (NCBI GI No. 5931761). Shown in Table 6 are the BLAST results for individual ESTs (“EST”), the sequences of the entire cDNA inserts comprising the indicated cDNA clones (“FIS”), sequences of contigs assembled from two or more ESTs (“Contig”), sequences of contigs assembled from an FIS and one or more ESTs (“Contig*”), or sequences encoding the entire protein derived from an FIS, a contig, or an FIS and PCR (“CGS”):

TABLE 6
BLAST Results for Sequences Encoding Polypeptides Homologous
to ALS Small Subunit

| | | BLAST pLog Score |
|---|---|---|
| Clone | Status | NCBI GI No. 5931761 |
| cen3n.pk0112.c11 | FIS | 132.00 |
| p0094.csstl72ra (FIS) | CGS | 150.00 |
| rl0n.pk117.a16 | FIS | 145.00 |
| wdk2c.pk015.a13 | FIS | 52.70 |

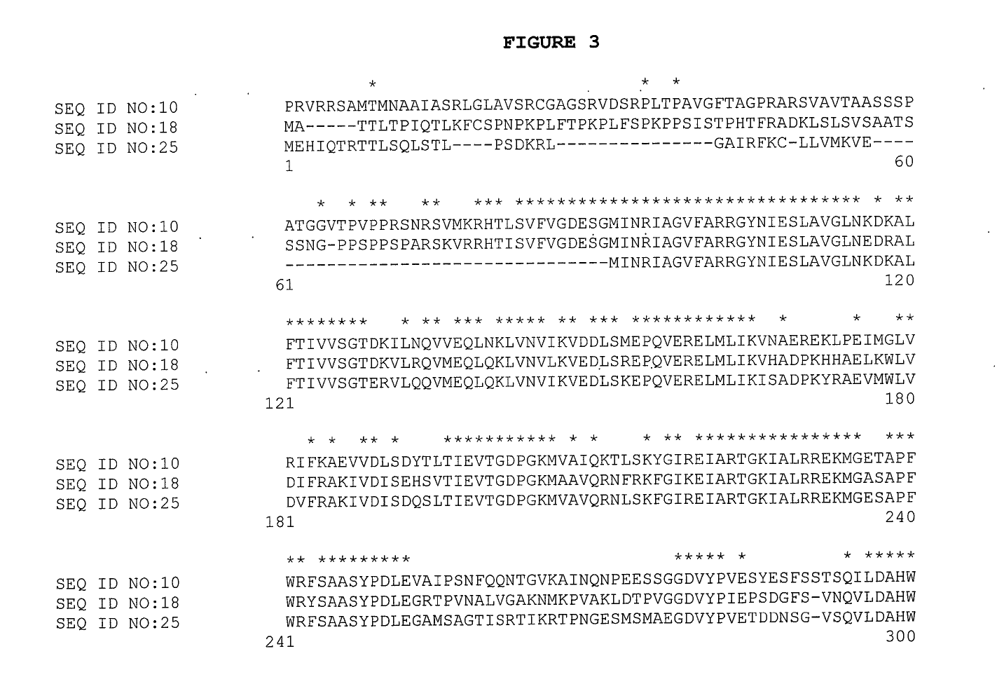

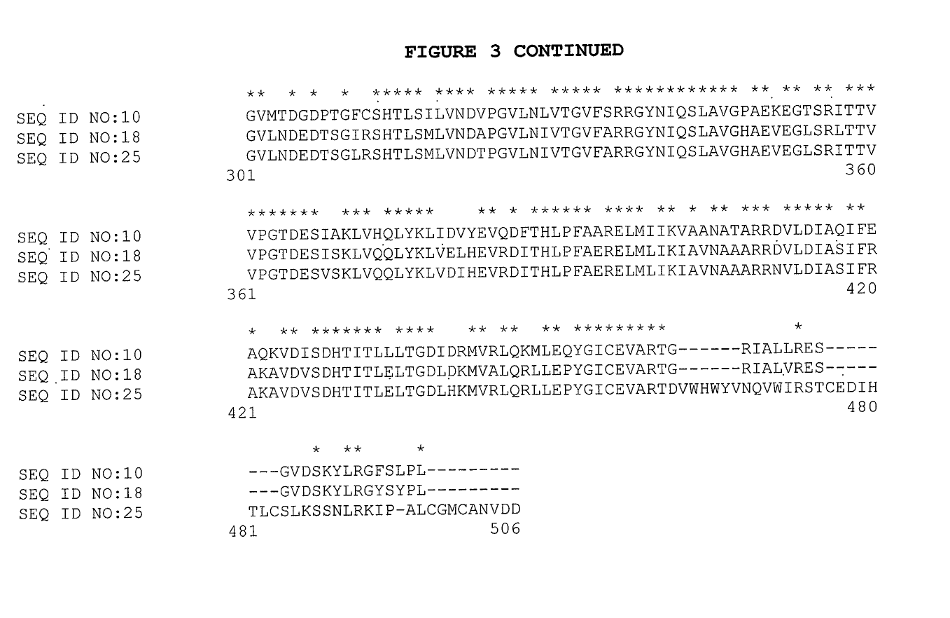

FIG. 3 presents an alignment of the amino acid sequences set forth in SEQ ID NOs:10 and 18 and the Nicotiana plumbaginifolia sequence (NCBI GI No. 5931761; SEQ ID NO:25). The data in Table 7 represents a calculation of the percent identity of the amino acid sequences set forth in SEQ ID NOs:10 and 18 and the Nicotiana plumbaginifolia sequence (NCBI GI No. 5931761; SEQ ID NO:25).

TABLE 7
Percent Identity of Amino Acid Sequences Deduced From the
Nucleotide Sequences of cDNA Clones Encoding Polypeptides
Homologous to ALS Small Subunit

| | Percent Identity to |
|---|---|
| SEQ ID NO. | NCBI GI No. 5931761; SEQ ID NO: 25 |
| 10 | 59.5 |
| 18 | 69.0 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode all or a substantial portion of an ALS small subunit. These sequences represent the first monocot (corn, rice, and wheat) and soybean sequences encoding ALS small subunit known to Applicant.

### Example 12

### Expression of Chimeric Genes in Monocot Cells

A chimeric gene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding the instant polypeptide, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 13

### Expression of Chimeric Genes in Dicot Cells

A seed-specific expression cassette composed of the promoter and transcription terminator from the gene encoding the D subunit of the seed storage protein phaseolin from the bean Phaseolus vulgaris (Doyle et al. (1986) J Biol. Chem. 261:9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic™ PDS1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptide and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 14

### Expression of Chimeric Genes in Microbial Cells

The cDNAs encoding the instant polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% low melting agarose gel. Buffer and agarose contain 10 μg/ml ethidium bromide for visualization of the DNA fragment. The fragment can then be purified from the agarose gel by digestion with GELase™ (Epicentre Technologies, Madison, Wis.) according to the manufacturer's instructions, ethanol precipitated, dried and resuspended in 20 μL of water. Appropriate oligonucleotide adapters may be ligated to the fragment using T4 DNA ligase (New England Biolabs (NEB), Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptide are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

What is claimed is:

1. An isolated polynucleotide comprising:
(a) a first nucleotide sequence encoding a first polypeptide comprising at least 35 amino acids, wherein the amino acid sequence of the first polypeptide and the amino acid sequence of SEQ ID NO:8 have at least 85% identity based on the Clustal alignment method,
(b) a second nucleotide sequence encoding a second polypeptide comprising at least 100 amino acids, wherein the amino acid sequence of the second polypeptide and the amino acid sequence of SEQ ID NO:4, SEQ ID NO:20, or SEQ ID NO:22 have at least 80% identity based on the Clustal alignment method,
(c) a third nucleotide sequence encoding a third polypeptide comprising at least 140 amino acids, wherein the amino acid sequence of the third polypeptide and the amino acid sequence of SEQ ID NO:14 have at least 80% identity based on the Clustal alignment method,
(d) a fourth nucleotide sequence encoding a fourth polypeptide comprising at least 150 amino acids, wherein the amino acid sequence of the fourth polypeptide and the amino acid sequence of SEQ ID NO:10 or SEQ ID NO:12 have at least 80% identity based on the Clustal alignment method,
(e) a fifth nucleotide sequence encoding a fifth polypeptide comprising at least 200 amino acids, wherein the amino acid sequence of the fifth polypeptide and the amino acid sequence of SEQ ID NO:6 have at least 80% identity based on the Clustal alignment method,
(f) a sixth nucleotide sequence encoding a sixth polypeptide comprising at least 200 amino acids, wherein the amino acid sequence of the sixth polypeptide and the amino acid sequence of SEQ ID NO:18 have at least 85% identity based on the Clustal alignment method,
(g) a seventh nucleotide sequence encoding a seventh polypeptide comprising at least 250 amino acids, wherein the amino acid sequence of the seventh polypeptide and the amino acid sequence of SEQ ID NO:16 have at least 80% identity based on the Clustal alignment method, or
(h) the complement of the first, second, third, fourth, fifth, sixth, or seventh nucleotide sequence, wherein the complement and the first, second, third, fourth, fifth, sixth, or seventh nucleotide sequence contain the same number of nucleotides and are 100% complementary.

2. The polynucleotide of claim 1, wherein the first polypeptide comprises at least 100 amino acids.

3. The polynucleotide of claim 1, wherein the amino acid sequence of the second polypeptide and the amino acid sequence of SEQ ID NO:4, SEQ ID NO:20, or SEQ ID NO:22 have at least 85% identity based on the Clustal alignment method, wherein the amino acid sequence of the third polypeptide and the amino acid sequence of SEQ ID NO:14 have at least 85% identity based on the Clustal alignment method, wherein the amino acid sequence of the fourth polypeptide and the amino acid sequence of SEQ ID NO:10 or SEQ ID NO:12 have at least 85% identity based on the Clustal alignment method, wherein the amino acid sequence of the fifth polypeptide and the amino acid sequence of SEQ ID NO:6 have at least 85% identity based on the Clustal alignment method, and wherein the amino acid sequence of the seventh polypeptide and the amino acid sequence of SEQ ID NO:16 have at least 85% identity based on the Clustal alignment method.

4. The polynucleotide of claim 1, wherein the amino acid sequence of the first polypeptide and the amino acid sequence of SEQ ID NO:8 have at least 90% identity based on the Clustal alignment method, wherein the amino acid sequence of the second polypeptide and the amino acid sequence of SEQ ID NO:4, SEQ ID NO:20, or SEQ ID NO:22 have at least 90% identity based on the Clustal alignment method, wherein the amino acid sequence of the third polypeptide and the amino acid sequence of SEQ ID NO:14 have at least 90% identity based on the Clustal alignment method, wherein the amino acid sequence of the fourth polypeptide and the amino acid sequence of SEQ ID NO:10 or SEQ ID NO:12 have at least 90% identity based on the Clustal alignment method, wherein the amino acid sequence of the fifth polypeptide and the amino acid sequence of SEQ ID NO:6 have at least 90% identity based on the Clustal alignment method, wherein the amino acid sequence of the sixth polypeptide and the amino acid sequence of SEQ ID NO:18 have at least 90% identity based on the Clustal alignment method, and wherein the amino acid sequence of the seventh polypeptide and the amino acid sequence of SEQ ID NO:16 have at least 90% identity based on the Clustal alignment method.

5. The polynucleotide of claim 1, wherein the amino acid sequence of the first polypeptide and the amino acid sequence of SEQ ID NO:8 have at least 95% identity based on the Clustal alignment method, wherein the amino acid sequence of the second polypeptide and the amino acid sequence of SEQ ID NO:4, SEQ ID NO:20, or SEQ ID NO:22 have at least 95% identity based on the Clustal alignment method, wherein the amino acid sequence of the third polypeptide and the amino acid sequence of SEQ ID NO:14 have at least 95% identity based on the Clustal alignment method, wherein the amino acid sequence of the fourth polypeptide and the amino acid sequence of SEQ ID NO:10 or SEQ ID NO:12 have at least 95% identity based on the Clustal alignment method, wherein the amino acid sequence of the fifth polypeptide and the amino acid sequence of SEQ ID NO:6 have at least 95% identity based on the Clustal alignment method, wherein the amino acid sequence of the sixth polypeptide and the amino acid sequence of SEQ ID NO:18 have at least 95% identity based on the Clustal alignment method, and wherein the amino acid sequence of the seventh polypeptide and the amino acid sequence of SEQ ID NO:16 have at least 95% identity based on the Clustal alignment method.

6. The isolated polynucleotide of claim 1, wherein the first polypeptide comprises the amino acid sequence of SEQ ID NO:8, wherein the second polypeptide comprises the amino acid sequence of SEQ ID NO:4, SEQ ID:20, or SEQ ID NO:22, wherein the third polypeptide comprises the amino acid sequence of SEQ ID NO:14, wherein the fourth polypeptide comprises the amino acid sequence of SEQ ID NO:10 or SEQ ID NO:12, wherein the fifth polypeptide comprises the amino acid sequence of SEQ ID NO:6, wherein the sixth polypeptide comprises the amino acid sequence of SEQ ID NO:18, and wherein the seventh polypeptide comprises the amino acid sequence of SEQ ID NO:16.

7. The isolated polynucleotide of claim 1, wherein the first nucleotide sequence comprises the nucleotide sequence of SEQ ID NO:7, wherein the second nucleotide sequence comprises the nucleotide sequence of SEQ ID NO:3, SEQ ID NO:19 or SEQ ID NO:21, wherein the third nucleotide sequence comprises the nucleotide sequence of SEQ ID NO:13, wherein the fourth nucleotide sequence comprises the nucleotide sequence of SEQ ID NO:9 or SEQ ID NO:11, wherein the fifth nucleotide sequence comprises the nucleotide sequence of SEQ ID NO:5, wherein the sixth nucleotide sequence comprises the nucleotide sequence of SEQ ID NO:17, and wherein the seventh nucleotide sequence comprises the nucleotide sequence of SEQ ID NO:15.

8. The isolated polynucleotide of claim 1, wherein the first, second, third, fourth, fifth, sixth, and seventh polypeptides are acetolactate synthase small subunits.

9. A chimeric gene comprising the polynucleotide of claim 1 operably linked to a regulatory sequence.

10. A vector comprising the polynucleotide of claim 1.

11. An isolated polynucleotide fragment comprising a nucleotide sequence comprised by the polynucleotide of claim 1, wherein the nucleotide sequence contains at least 30 nucleotides.

12. The fragment of claim 11, wherein the nucleotide sequence contains at least 40 nucleotides.

13. The fragment of claim 11, wherein the nucleotide sequence contains at least 60 nucleotides.

14. An isolated polypeptide comprising:
(a) a first amino acid sequence comprising at least 35 amino acids, wherein the first amino acid sequence and the amino acid sequence of SEQ ID NO:8 have at least 85% identity based on the Clustal alignment method,
(b) a second amino acid sequence comprising at least 100 amino acids, wherein the second amino acid sequence and the amino acid sequence of SEQ ID NO:4, SEQ ID NO:20 or SEQ ID NO:22 have at least 80% identity based on the Clustal alignment method,
(c) a third amino acid sequence comprising at least 140 amino acids, wherein the third amino acid sequence and the amino acid sequence of SEQ ID NO:14 have at least 80% identity based on the Clustal alignment method,
(d) a fourth amino acid sequence comprising at least 150 amino acids, wherein the fourth amino acid sequence and the amino acid sequence of SEQ ID NO:10 or SEQ ID NO:12 have at least 80% identity based on the Clustal alignment method,
(e) a fifth amino acid sequence comprising at least 200 amino acids, wherein the fifth amino acid sequence and the amino acid sequence of SEQ ID NO:6 have at least 80% identity based on the Clustal alignment method,
(f) a sixth amino acid sequence comprising at least 200 amino acids, wherein the sixth amino acid sequence and the amino acid sequence of SEQ ID NO:18 have at least 85% identity based on the Clustal alignment method, or
(g) a seventh amino acid sequence comprising at least 250 amino acids, wherein the seventh amino acid sequence and the amino acid sequence of SEQ ID NO:16 have at least 80% identity based on the Clustal alignment method.

15. The polypeptide of claim 14, wherein the first amino acid sequence comprises at least 100 amino acids.

16. The polypeptide of claim 14, wherein the second amino acid sequence and the amino acid sequence of SEQ ID NO:4, SEQ ID NO:20, or SEQ ID NO:22 have at least 85% identity based on the Clustal alignment method, wherein the third amino acid sequence and the amino acid sequence of SEQ ID NO:14 have at least 85% identity based on the Clustal alignment method, wherein the fourth amino acid sequence and the amino acid sequence of SEQ ID NO:10 or SEQ ID NO:12 have at least 85% identity based on the Clustal alignment method, wherein the fifth amino acid sequence and the amino acid sequence of SEQ ID NO:6 have at least 85% identity based on the Clustal alignment method, and wherein the seventh amino acid sequence and the amino acid sequence of SEQ ID NO:16 have at least 85% identity based on the Clustal alignment method.

17. The polypeptide of claim 14, wherein the first amino acid sequence and the amino acid sequence of SEQ ID NO:8 have at least 90% identity based on the Clustal alignment method, wherein the second amino acid sequence and the amino acid sequence of SEQ ID NO:4, SEQ ID NO:20, or SEQ ID NO:22 have at least 90% identity based on the Clustal alignment method, wherein the third amino acid sequence and the amino acid sequence of SEQ ID NO:14 have at least 90% identity based on the Clustal alignment method, wherein the fourth amino acid sequence and the amino acid sequence of SEQ ID NO:10 or SEQ ID NO:12 have at least 90% identity based on the Clustal alignment method, wherein the fifth amino acid sequence and the amino acid sequence of SEQ ID NO:6 have at least 90% identity based on the Clustal alignment method, wherein the sixth amino acid sequence and the amino acid sequence of SEQ ID NO:18 have at least 90% identity based on the Clustal alignment method, and wherein the seventh amino acid sequence and the amino acid sequence of SEQ ID NO:16 have at least 90% identity based on the Clustal alignment method.

18. The polypeptide of claim 14, wherein the first amino acid sequence and the amino acid sequence of SEQ ID NO:8 have at least 95% identity based on the Clustal alignment method, wherein the second amino acid sequence and the amino acid sequence of SEQ ID NO:4, SEQ ID NO:20, or SEQ ID NO:22 have at least 95% identity based on the Clustal alignment method, wherein the third amino acid sequence and the amino acid sequence of SEQ ID NO:14 have at least 95% identity based on the Clustal alignment method, wherein the fourth amino acid sequence and the amino acid sequence of SEQ ID NO:10 or SEQ ID NO:12 have at least 95% identity based on the Clustal alignment method, wherein the fifth amino acid sequence and the amino acid sequence of SEQ ID NO:6 have at least 95% identity based on the Clustal alignment method, wherein the sixth amino acid sequence and the amino acid sequence of SEQ ID NO:18 have at least 95% identity based on the Clustal alignment method, and wherein the seventh amino acid sequence and the amino acid sequence of SEQ ID NO:16 have at least 95% identity based on the Clustal alignment method.

19. The polypeptide of claim 14, wherein the first amino acid sequence comprises the amino acid sequence of SEQ ID NO:8, wherein the second amino acid sequence comprises the amino acid sequence of SEQ ID NO:4, SEQ ID:20, or SEQ ID NO:22, wherein the third amino acid sequence comprises the amino acid sequence of SEQ ID NO:14, wherein the fourth amino acid sequence comprises the amino acid sequence of SEQ ID NO:10 or SEQ ID NO:12, wherein the fifth amino acid sequence comprises the amino acid sequence of SEQ ID NO:6, wherein the sixth amino acid sequence comprises the amino acid sequence of SEQ ID NO:18, and wherein the seventh amino acid sequence comprises the amino acid sequence of SEQ ID NO:16.

20. The polypeptide of claim 14, wherein the polypeptide is an acetolactate synthase small subunit.

21. A method for transforming a cell comprising introducing the polynucleotide of claim 1 into a cell.

22. The cell produced by the method of claim 21.

23. A method for producing a transgenic plant comprising transforming a plant cell with the polynucleotide of claim 1 and regenerating a plant from the transformed plant cell.

24. The transgenic plant produced by the method of claim 23.

25. A seed obtained from the transgenic plant of claim 24.


SEQUENCE LISTING (43 sequences)

SEQ ID NO: 1 - DNA, 611 bp, Zea mays
  unsure (61)
  unsure (328)
  unsure (331)
  unsure (357)
  unsure (361)
  unsure (364)
agcattcggcacgagcgaaaatccacttacatctgtaaataggacagttaatggcagtttnggtcaaccatccaatgctgggggtgatgtttatcctgtggaatcttacgagagcttatcagtgaaccatgtacttgatgctcattggggtgttctggatgatgatgatgcgactggacttcgctcgcatactctctccatccttgtgaatgactgtcctggtgtcctcaacattgtaacaggagtctttgctcgcaggggctacaatatacagagccttgctgttggcccagctgagaaggaaggcatttcgcggattacaacagtngtncctggtactgttgaatccattgaganagtnagntcagccagctttacaagcttattgatgtgtaacgaaagttccatgacattacccactcaccttttgctgaaagggaacttatccttatctaaggtttctgtcaacactgctgctcggaaggaaatcctacatattgctcaaatcttccgagcaaaacctgttgatgtttctgaccacacagtaacgcttcagcttactggagatcttgacaagatggttgacactacaaaggttattggagccatat

SEQ ID NO: 2 - protein, 203 aa, Zea mays
  UNSURE (20)
  UNSURE (119)
  UNSURE (121)
AFGTSENPLTSVNRTVNGSXGQPSNAGGDVYPVESYESLSVNHVLDAHWGVLDDDDATGLRSHTLSILVNDCPGVLNIVTGVFARRGYNIQSLAVGPAEKEGISRITTVVPGTVESIEXVXSASFTSLLMCNESSMTLPTHLLLKGNLSLSKVSVNTAARKEILHIAQIFRAKPVDVSDHTVTLQLTGDLDKMVDTTKVIGAI

SEQ ID NO: 3 - DNA, 470 bp, Zea mays
  unsure (106)..(107)
  unsure (130)
  unsure (164)
  unsure (166)
  unsure (172)
  unsure (174)
  unsure (193)
  unsure (199)
  unsure (287)
  unsure (296)
  unsure (308)
  unsure (358)
  unsure (391)
  unsure (447)
  unsure (452)
  unsure (454)
accaagtcattgagcagctcaataagctcgtcaacgttcatagtgttgaagatctatctaaagaacctcaggttgaaagagagctgatgcttataaagctaaacgnngaacctgatcagcgccctgaggncatggttttagttgacattttcagagcaaaagtngntgatananctgagaaaacacttaccanagaggnagctggagatcctggcaaaattgctgcagtgcagaggaatctaaggaaattcggcatcaaagaaatttgcaggacaggaaaaattgcnttgagacgngaaaagattggngcaacagcccgtttctggcgattttctgctgcttcttatccagaccttanagaggcattaccaaaaaaaccgcttacatctgnaaataagacagtgaatggcagttttgttcgaccatccaatgctgggggtgatgttnatccngnggaatcttacgagagc

SEQ ID NO: 4 - protein, 156 aa, Zea mays
  UNSURE (35)
  UNSURE (43)
  UNSURE (55)
  UNSURE (57)..(58)
  UNSURE (64)
  UNSURE (66)
  UNSURE (119)
  UNSURE (130)
  UNSURE (149)
  UNSURE (151)
QVIEQLNKLVNVHSVEDLSKEPQVERELMLIKLNXEPDQRPEXMVLVDIFRAKVXDXXEKTLTXEXAGDPGKIAAVQRNLRKFGIKEICRTGKIALRREKIGATARFWRFSAASYPDLXEALPKKPLTSXNKTVNGSFVRPSNAGGDVXPXESYES

SEQ ID NO: 5 - DNA, 1297 bp, Zea mays
caaccaagtcattgagcagctcaataagctcgtcaacgttcatagtgttgaagatctatctaaagaacctcaggttgaaagagagctgatgcttataaagctaaacgttgaacctgatcagcgccctgaggtcatggttttagttgacattttcagagcaaaagttgttgatatatctgagaaaacacttaccatagaggtagctggagatcctggcaaaattgctgcagtgcagaggaatctaaggaaattcggcatcaaagaaatttgcaggacaggaaaaattgctttgagacgtgaaaagattggtgcaacagcccgtttctggcgattttctgctgcttcttatccagaccttatagaggcattaccaaaaaaaccgcttacatctgtaaataagacagtgaatggcagttttgttcgaccatccaatgctgggggtgatgtttatcctgtggaatcttacgagagctcagctaaccaagtacttgatgctcactggggtgttctggatgatgatgatgcaactggactttgctcgcataccctctccatccttgtgaatgattgtcctggtgttctcaacattgtaacaggagtctttgctcgcaggggctacaatatacagagccttgctgttggctcagctgagaaggaaggcatttcacgtattacaacagttgttcctggtactgttgaatccattgggaagttagttcagcagctttacaagcttattgatgtgcatgaagttcatgacattacccactcaccttttgctgaaagggagctgatgcttattaaggtttctgtaaacactgctgctcggagggaaattctagatattgctgaaatcttccgagcaaaacctattgacgtttctgaccatacagtaacccttcagcttactggagatcttgacaagatggttgcactacaaaggttattagagccatatggcatctgcgaggtcgccagaactggacgagtggcactggtccgtgaatcgaaggtcgactccaagtacctccgcggttactctcttccattgtagcctggcatttgtgattggtggtggacccgataaagagcttggtttgttgttatagatcagaccatctcgcgctgggatgtgttgtgcaattacaggacttgtttctttcatgttgtgaactccctggcctgcgagttcaataatgcccctttttaatgcgcgttgaggttgcatatgtatcccatcgactgtccgaataagatgcatagcataactgtttacttcaaaaaaaaaaaaaaaaaa

SEQ ID NO: 6 - protein, 350 aa, Zea mays
NQVIEQLNKLVNVHSVEDLSKEPQVERELMLIKLNVEPDQRPEVMVLVDIFRAKVVDISEKTLTIEVAGDPGKIAAVQRNLRKFGIKEICRTGKIALRREKIGATARFWRFSAASYPDLIEALPKKPLTSVNKTVNGSFVRPSNAGGDVYPVESYESSANQVLDAHWGVLDDDDATGLCSHTLSILVNDCPGVLNIVTGVFARRGYNIQSLAVGSAEKEGISRITTVVPGTVESIGKLVQQLYKLIDVHEVHDITHSPFAERELMLIKVSVNTAARREILDIAEIFRAKPIDVSDHTVTLQLTGDLDKMVALQRLLEPYGICEVARTGRVALVRESKVDSKYLRGYSLPL

SEQ ID NO: 7 - DNA, 547 bp, Zea mays
  unsure (28)
  unsure (37)
  unsure (358)
  unsure (383)
  unsure (394)
  unsure (400)
  unsure (468)
  unsure (506)
  unsure (536)..(537)
  unsure (544)
gccgtttagcaatgaccatgaacgctgngatcgcctnccgactcggcctcgccgtctccaggtgtggggctgggtcccgagtggacagccggccgctgacgccggcagtgggtttcacggcggggccgagggcgcgctcagtagccgtcaccgccgcctcctcttctccggcgaccggtggcgtgacgccggtgccaccccgctcgaatcgctcggttatgaagcgtcacacgctatcaagtttttgttggtgatgaaagtgggatgatcaatcgaatttgctggggtttttgctagaagaggatataacatcgagtcattggctgttgggttgaacaaggataaagcattatttacnatagtagttgtcaaggaacagacanaatattaaacnaagtcngtagagcaactaaacaaagcttgttaatgtcataaaggttgatgatttatcaatggaaccacaagttnaaagagaacttatgcttataaaattaaatgctaacganaaaaagtacctgagataatgggtttggttnnatttttnaaa

SEQ ID NO: 8 - protein, 43 aa, Zea mays
  UNSURE (10)
KFLLVMKVGXSIEFAGVFARRGYNIESLAVGLNKDKALFTIVV

SEQ ID NO: 9 - DNA, 1813 bp, Zea mays
ccacgcgtccgccgttcagcaatgaccatgaacgctgcgatcgcctcccgactcggcctcgccgtctccaggtgtggggctgggtcccgagtggacagccggccgctgacgccggcagtgggtttcacggcggggccgagggcgcgctcagtagccgtcaccgccgcctcctcttctccggcgaccggtggcgtgacgccggtgccaccccgctcgaatcgctcggttatgaagcgtcacacgctatcagtttttgttggtgatgaaagtgggatgatcaatcgaattgctggggtttttgctagaagaggatataacatcgagtcattggctgttgggttgaacaaggataaagcattatttacaatagtagtgtcaggaacagacaaaatattaaaccaggtcgtagagcaactaaacaagcttgttaatgtcataaaggttgatgatttatcaatggaaccacaagttgaaagagaacttatgcttataaaagtaaatgcagagcgagaaaagttacctgagataatgggtttggttcgcattttcaaagcagaagtggttgatctttcagactacacactaactattgaggtaactggagatcctggaaagatggttgcaatacagaagactctgagcaaatatgggatcagagaaattgctagaactggcaagatagctttgcgccgtgaaaaaatgggagaaactgctccattttggaggttctctgcagcttcttatccggatctcgaagtggcaataccttcaaatttccagcaaaacactggtgtgaaggcaatcaatcagaatccggaagaatcttcagggggtgatgtttatccagtggaatcttatgaaagcttctcatcaacaagtcaaattctggatgctcattggggtgttatgactgatggcgatccaacagggttttgttcacatactctatcaattcttgtgaatgatgtccctggagttctcaatcttgtaacaggtgtattctccagaaggggctacaatattcagagtcttgctgttggcccagctgaaaaagaaggaacttctcgcatcactactgttgttcctggaactgatgaatccattgccaagctagtacatcaactgtacaagctcattgatgtttatgaagttcaggattttacccacttaccatttgctgctagagagttaatgatcataaaggtcgcggcaaatgctacagctcgaagggatgtcttagatattgctcagatttttgaggcacagaaagttgacatatcagaccacacaattacactactgctcaccggagacattgacagaatggttagattgcaaaagatgctagagcagtatggcatctgtgaggttgcacggacaggccggattgctctgctccgagagtctggagttgactccaagtacctccgcgggttttccctcccgctgtaattctccatttccagacattacactgcgcgatttcaggtcgccacggtcattttgacttctgagaatggagctggaggaatcattcaaaaatccaagggtgagaactgtagaaataatagctgttaagtttcttgcttctgcaagtatcacgcagcacatggggagagatgaagtatcatacacaacaatatgtctgcagtcagaaacgatcgatgaggggtccaatatttttttcactacacatgttgtatacgtacactggtagtatcctagttgaaggaaacgcaatatatatatattcactaaaaaaaaaaaaaaaaaaaaaaaaaaaaaaaaaaaaaaaaaaaaaaaaaaaaaaaaa

SEQ ID NO: 10 - protein, 483 aa, Zea mays
PRVRRSAMTMNAAIASRLGLAVSRCGAGSRVDSRPLTPAVGFTAGPRARSVAVTAASSSPATGGVTPVPPRSNRSVMKRHTLSVFVGDESGMINRIAGVFARRGYNIESLAVGLNKDKALFTIVVSGTDKILNQVVEQLNKLVNVIKVDDLSMEPQVERELMLIKVNAEREKLPEIMGLVRIFKAEVVDLSDYTLTIEVTGDPGKMVAIQKTLSKYGIREIARTGKIALRREKMGETAPFWRFSAASYPDLEVAIPSNFQQNTGVKAINQNPEESSGGDVYPVESYESFSSTSQILDAHWGVMTDGDPTGFCSHTLSILVNDVPGVLNLVTGVFSRRGYNIQSLAVGPAEKEGTSRITTVVPGTDESIAKLVHQLYKLIDVYEVQDFTHLPFAARELMIIKVAANATARRDVLDIAQIFEAQKVDISDHTITLLLTGDIDRMVRLQKMLEQYGICEVARTGRIALLRESGVDSKYLRGFSLPL

SEQ ID NO: 11 - DNA, 515 bp, Oryza sativa
  unsure (462)
  unsure (481)
cttacagttgaaccagatcagcgtcctgaggtcatggttttagttgatattttccgagcgaaagttgttgatatttcggagaacacccttaccatcgaggtaactggagatcctggcaaaattgttgctgtgcaaaggaacctcagcaaatttgggataaaagaaatttgtagaacgggaaaaattgctttgagacgtgaaaaaattggagcaactgcccgcttctggggattttctgctgcttcttacccagatctcatagaggcattgcccaaaaattctcttcttacttctgtaaataagacagtcaatggaagttttgatcaaccatccaatgctgggggcgatgtctatcctgtggaaccttatgagggttcatccatgaaccaagtacttgatgctcactggggcgtccttgatgatgaagatcaagtggacttcgatcacatactcaaccatccntgtcaatgattgccctggngttccaacattgtacaggggccttgctccaaagg

SEQ ID NO: 12 - protein, 156 aa, Oryza sativa
LTVEPDQRPEVMVLVDIFRAKVVDISENTLTIEVTGDPGKIVAVQRNLSKFGIKEICRTGKIALRREKIGATARFWGFSAASYPDLIEALPKNSLLTSVNKTVNGSFDQPSNAGGDVYPVEPYEGSSMNQVLDAHWGVLDDEDQVDFDHILNHPCQ

SEQ ID NO: 13 - DNA, 484 bp, Oryza sativa
  unsure (350)
  unsure (419)
  unsure (424)
  unsure (436)
  unsure (440)
  unsure (463)
  unsure (476)
cttacactaaaccgtggggctcaacaaggacaaggccatgttcaccattgtcgtctccggcacggacagggtgctcaaccaagtcatcgagcagctcaacaagcttgtcaacgtcttgaatgtggaagatctatctaaggagccacaggttgaaagagagctgatgcttataaaaattaatgttgaaccagatcagcgtcctgaggtcatggttttagttgatattttccgagcgaaagttgttgatatttcggagaacacccttaccatcgaggtaactgggagatcctgggcaaaattgttgctgtgcaaaggaacctcagcaaatttgggaaaaaagaaatttgtanaacgggaaaaattggctttgagacgtgaaaaaatttgggagcaactggccgcctccgggggaatttccngctgnctccttaccaanatcncataaaaggaatggcccaaaaantcccctcctaacntccggaaa

SEQ ID NO: 14 - protein, 144 aa, Oryza sativa
  UNSURE (114)
  UNSURE (137)..(138)
  UNSURE (142)
  UNSURE (144)
TVGLNKDKAMFTIVVSGTDRVLNQVIEQLNKLVNVLNVEDLSKEPQVERELMLIKINVEPDQRPEVMVLVDIFRAKVVDISENTLTIEVTGRSWAKLLLCKGTSANLGKKKFVXREKLALRREKIWEQLAASGGISXXLLTXIX

SEQ ID NO: 15 - DNA, 1435 bp, Oryza sativa
gcacgagcttacactaaaccgtggggctcaacaaggacaaggccatgttcaccattgtcgtctccggcacggacagggtgctcaaccaagtcatcgagcagctcaacaagcttgtcaacgtcttgaatgtggaagatctatctaaggagccacaggttgaaagagagctgatgcttataaaaattaatgttgaaccagatcagcgtcctgaggtcatggttttagttgatattttccgagcgaaagttgttgatatttcggagaacacccttaccatcgaggtaactggagatcctggcaaaattgttgctgtgcaaaggaacctcagcaaatttgggataaaagaaatttgtagaacgggaaaaattgctttgagacgtgaaaaaattggagcaactgcccgcttctggggattttctgctgcttcttacccagatctcatagaggcattgcccaaaaattctcttcttacttctgtaaataagacagtcaatggaagttttgatcaaccatccaatgctgggggcgatgtctatcctgtggaaccttatgagggttcatccatgaaccaagtacttgatgctcactggggcgtccttgatgatgaagattcaagtggacttcgatcacatactctatccatccttgtcaatgattgccctggtgttctcaacattgttacaggggtctttgctcgcagaggctacaatatacagagtcttgctgtaggcccagctgaaaagtcaggcctttcgcgtattacaacagttgctcctggaacagatgaatccattgagaagttagttcagcagcttaacaaacttgttgatgtgcatgaggttcaagatataactcacttgccttttgctgaaagagaacttatgcttatcaaggtttctgtgaacactgctgctcggagagacatactagatattgctgaaatcttccgggcaaaatctgttgatgtttctgatcacactgttacgttacagcttactggggatctcgacaagatggttgcattacaaaggctgttggagccttatggcatctgtgaggtcgccagaacagggcgagtggcgctggtccgcgaatccggtgtcgattccaagtaccttcgtggctactcctttccgttgtaatcccaggtcttgtgagaagaaaggacagtaataaaatgcttggtcggttggttgctacctgttacagcagagtgttgtagtagagtgttgtagtcagattccgttcgttcagttatgttgtttgttatgatgctgttcttttgttgttgtttaccttcctctctgtaatgtgccaatccgctggcttcttgtccagtaaagatcatgatgcaagagttgagcctatgttttctaaaaaaaaaaaaaaaaaaaaaaaaaaaaaaaaaaaaaaaaaaaaaaaaaaaaaaaaaaaa

SEQ ID NO: 16 - protein, 365 aa, Oryza sativa
MFTIVVSGTDRVLNQVIEQLNKLVNVLNVEDLSKEPQVERELMLIKINVEPDQRPEVMVLVDIFRAKVVDISENTLTIEVTGDPGKIVAVQRNLSKFGIKEICRTGKIALRREKIGATARFWGFSAASYPDLIEALPKNSLLTSVNKTVNGSFDQPSNAGGDVYPVEPYEGSSMNQVLDAHWGVLDDEDSSGLRSHTLSILVNDCPGVLNIVTGVFARRGYNIQSLAVGPAEKSGLSRITTVAPGTDESIEKLVQQLNKLVDVHEVQDITHLPFAERELMLIKVSVNTAARRDILDIAEIFRAKSVDVSDHTVTLQLTGDLDKMVALQRLLEPYGICEVARTGRVALVRESGVDSKYLRGYSFPL

SEQ ID NO: 17 - DNA, 1721 bp, Glycine max
gcacgaggaaagcagtagtagaagaagaaaacacgagttacgcacagtcccggtgccgccaccaccaccattggtttcagtcaactaatggcaaccactctcactccaatccaaaccctcaaattctgttctcccaatcccaaacctctcttcactcccaaacctctcttctctcccaaacctccttccatctccacacctcacaccttccgcgccgataaactctctctctccgtctccgccgccacctcctcctccaacggtcctccctctcctccctcccccgctcgctcaaaggttcgtcgacacacgatttccgtgttcgtcggcgatgagagcggaatgataaaccggattgccggcgtgttcgctaggagaggatacaacatcgaatccctcgcggttggcctcaatgaggacagggcgctcttcaccatcgtcgtgtcagggaccgataaggtgctgcgccaagtcatggagcagcttcagaaactcgtcaatgtcttaaaggtagaggatctttcgagggaaccacaggtggaacgtgaactgatgctcataaaagtgcatgcggatccgaaacaccatgcggagttgaagtggttggtggacatcttcagagctaagattgtggatatctcggaacattcggtgacaattgaggtaactggagatccagggaagatggccgcggttcaaagaaatttccgcaagtttggaattaaagaaatagccagaactggaaagattgcattaagaagggaaaagatgggtgcatctgctccattttggcggtattcagctgcttcttatccagatcttgaaggaagaacacctgttaatgcccttgtgggagcaaaaaatatgaaacctgttgccaaacttgatacacctgtggggggagatgtttatcctatagaaccatcagatggtttttcagtcaatcaagttcttgacgctcactggggtgtcctcaatgatgaagataccagtggaattcgatcacacacgttatccatgcttgtgaacgatgctcctggagttctaaacattgttacaggagtttttgctagaagaggctataacattcagagtttagctgtaggacatgcagaagttgaaggactttctcgacttacaactgtggttcctgggacagatgagtcaattagcaagttggtgcagcaactctataagctagtagagctacatgaggttcgtgatatcacccacttgccatttgctgagcgagaattgatgctaataaagattgctgtaaatgctgctgcacggcgtgatgtccttgatattgctagcattttccgggctaaagctgtcgatgtatctgatcacacaataactctggagcttactggagatttggacaagatggttgcattgcagagattgttagaaccatatggcatttgtgaggtggcacgaactgggcgaattgctctagtccgcgagtccggtgtggactccaagtacttgcgtggatactcttatcctttgtaatcactttcacctgggtggaagaacaatcaaagtcttaggattttaaagcttgtactttctcttaaacattgcctacggattcttgccttctcaactataatgttaagtctttcagtgatttgcaaatataggcttgggatgacatctgagatttttttttaaaaaaagttttcctcctcaaaaaaaaaaaaaaaaaaaaaaaa

SEQ ID NO: 18 - protein, 476 aa, Glycine max
MATTLTPIQTLKFCSPNPKPLFTPKPLFSPKPPSISTPHTFRADKLSLSVSAATSSSNGPPSPPSPARSKVRRHTISVFVGDESGMINRIAGVFARRGYNIESLAVGLNEDRALFTIVVSGTDKVLRQVMEQLQKLVNVLKVEDLSREPQVERELMLIKVHADPKHHAELKWLVDIFRAKIVDISEHSVTIEVTGDPGKMAAVQRNFRKFGIKEIARTGKIALRREKMGASAPFWRYSAASYPDLEGRTPVNALVGAKNMKPVAKLDTPVGGDVYPIEPSDGFSVNQVLDAHWGVLNDEDTSGIRSHTLSMLVNDAPGVLNIVTGVFARRGYNIQSLAVGHAEVEGLSRLTTVVPGTDESISKLVQQLYKLVELHEVRDITHLPFAERELMLIKIAVNAAARRDVLDIASIFRAKAVDVSDHTITLELTGDLDKMVALQRLLEPYGICEVARTGRIALVRESGVDSKYLRGYSYPL

SEQ ID NO: 19 - DNA, 417 bp, Triticum aestivum
  unsure (367)
  unsure (381)
gccaggagaggctacaacatcgagtcgctcgccgtggggcttaacaaggacaaggccctcttcaccatcgtcgtctccgggacggatagggtgctcaagcaagtcattgagcagctcaataagctcgtcaacgtcttgaatgttgaagatctatctaaggagcctcaggttgaaagagagctcatgcttataaaactcaatgttgaaccagatcaacgtgctgacgtcatgtttgtagctaatgttttcaagagcgaaagttgttgatatttctgagaacaagctaactctggaggtaactgggagatcctgggaaaatccgttgcggcacaaaagggaacctaaagaaaatttgggaattcaaaanaattttgtccaacngggaaaaattggcttttgagggaataaagggaccct

SEQ ID NO: 20 - protein, 135 aa, Triticum aestivum
  UNSURE (89)
  UNSURE (92)
  UNSURE (115)
  UNSURE (123)
ARRGYNIESLAVGLNKDKALFTIVVSGTDRVLKQVIEQLNKLVNVLNVEDLSKEPQVERELMLIKLNVEPDQRADVMFVANVFKSESCXYFXEQANSGGNWEILGKSVAAQKGTXRKFGNSKXFCPTGKNWLLRE

SEQ ID NO: 21 - DNA, 724 bp, Triticum aestivum
gcacgaggccaggagaggctacaacatcgagtcgctcgccgtggggcttaacaaggacaaggccctcttcaccatcgtcgtctccgggacggatagggtgctcaagcaagtcattgagcagctcaataagctcgtcaacgtcttgaatgttgaagatctatctaaggagcctcaggttgaaagagagctcatgcttataaaactcaatgttgaaccagatcaacgtgctgacgtcatgtttgtagctaatgttttcagagcgaaagttgttgatatttctgagaacagcctaactctggaggtaactggagatcctgggaaaatcgttgcggcacaaaggaacctaagaaaatttgggatcgaagaaatttgtcgaacgggaaaaattgctttgaggcaataaggaacctagcgctacagacgctacctgcaatgtgcaatgtgtatatccctccatattgcaccattgctccatttggcatcttcggtactaactgagaagagaagaactctagtagtagatatatgatacaccgttttcttagtccatggtttggtcgttgtagcggtgaaaaaataaagtgacatgcactatcatgtaagaacctgaactatactagttcaaacttgggaataaaagacaaacacaggtcttgtctaaaaaaaaaaaaaaaaaaaaaaaaaaaaaaaaaaaaaaaaaaaaaaaaaaaaaaaaaaaaaaaaa

SEQ ID NO: 22 - protein, 133 aa, Triticum aestivum
HEARRGYNIESLAVGLNKDKALFTIVVSGTDRVLKQVIEQLNKLVNVLNVEDLSKEPQVERELMLIKLNVEPDQRADVMFVANVFRAKVVDISENSLTLEVTGDPGKIVAAQRNLRKFGIEEICRTGKIALRQ

SEQ ID NO: 23 - DNA, 1861 bp, Nicotiana plumbaginifolia
ggattcggcacgagggcggctgtttcaactcccttcaacagctctctaaataaacccttcctttctctctattcctctattccccatttgggtttctcccaagttgttcaatttccaactttgaaaccaaaatttcttctttcaaagatcaaaacagttgtaatttctgcgccaaagctgatggaacatatacaaacgaggacgactctctcacaactttcgactcttccatcagacaaaagactaggcgccatacgattcaagtgtttgttggtgatgaaagtggaaatgatcaatcgtattgcgggagtctttgctcgaagagggtacaatattgagtctcttgctgttggtctaaacaaggacaaggctctttttactatagttgtttctggaactgaaagggtgttacagcaagttatggaacagttacaaaagcttgtaaatgtgatcaaggttgaagatctatccaaggagccacaagttgaacgagaattaatgcttattaaaatcagcgccgatccaaaataccgcgcagaggttatgtggttggtggacgttttcagggcaaaaattgtggatatatctgatcaatctctgactattgaggtaactggagatccagggaagatggtggctgttcagaggaacttaagtaaatttggaattagagaaattgctcgtactgggaagattgccttgagaagagaaaaaatgggggaatctgctcctttttggcggttttcagcagcatcatacccagatcttgaaggtgcaatgtctgctggtactatttcgaggacaatcaagaggacccctaatggagaatctatgtctatggctgagggagatgtctatcctgttgagacagatgacaactctggagtcagtcaagttcttgatgctcactggggtgttctcaatgatgaagatacaagtgggcttcgctcacatactttgtcaatgcttgtgaatgacactcctggagttctcaacatagtcaccggagtttttgctcgacgagggtataacatccaaagtttagctgttggacatgctgaagttgaggggctttctcgtattacaacggttgttcctggcacagatgagtcagttagcaagttggtgcagcaactatataagttggttgatattcatgaggttcgggatattactcacctcccatttgcggaaagagaactaatgttgataaagattgctgtgaatgctgcagcgcgccgcaatgttcttgacattgccagcattttcagagcaaaagctgttgatgtgtctgaccacactataactcttgagcttacaggagatttgcataagatggttcgtttgcagcggctactagagccttatggtatttgtgaggtagcgcgaacagacgtctggcactggtacgtgaatcaggtgtggattcgaagtacttgcgaggatattcataccctttgtagtctaaagtcctcgaacctaaggaaaatacctgctctttgcggaatgtgtgcgaacgtggatgactgaatgatacggagatggtctcgtgccccagctgtgaccctaccgctttcaagtcaagtttgcatgcttttagcttgaggtagttgcaagtttatgaaaatgaatagggtacaatttgaccattctatgaacaaaagcggacccagtctttaagatacaagagttttcagctcttttcttttcacctttattctttagcagagatagttcttcaataagcttcactgaaaatgattgacgtcgcgtcgtcatttgacttacaagtaaaatatgactagcatttttggtttttaaatctgaaagtgatatgaaacataaaaaaaaaaaaaaaaaa

SEQ ID NO: 24 - protein, 449 aa, Nicotiana plumbaginifolia
MEHIQTRTTLSQLSTLPSDKRLGAIRFKCLLVMKVEMINRIAGVFARRGYNIESLAVGLNKDKALFTIVVSGTERVLQQVMEQLQKLVNVIKVEDLSKEPQVERELMLIKISADPKYRAEVMWLVDVFRAKIVDISDQSLTIEVTGDPGKMVAVQRNLSKFGIREIARTGKIALRREKMGESAPFWRFSAASYPDLEGAMSAGTISRTIKRTPNGESMSMAEGDVYPVETDDNSGVSQVLDAHWGVLNDEDTSGLRSHTLSMLVNDTPGVLNIVTGVFARRGYNIQSLAVGHAEVEGLSRITTVVPGTDESVSKLVQQLYKLVDIHEVRDITHLPFAERELMLIKIAVNAAARRNVLDIASIFRAKAVDVSDHTITLELTGDLHKMVRLQRLLEPYGICEVARTDVWHWYVNQVWIRSTCEDIHTLCSLKSSNLRKIPALCGMCANVDD

SEQ ID NO: 25 - protein, 449 aa, Nicotiana plumbaginifolia; identical to SEQ ID NO: 24

SEQ ID NO: 26 - DNA, 20 bp, Artificial Sequence
  Description of Artificial Sequence:Synthetic Oligonucleotide
cccagtacgagcaatttctc

SEQ ID NO: 27 - DNA, 20 bp, Artificial Sequence
  Description of Artificial Sequence:Synthetic Oligonucleotide
gtggctccttggatagatct

SEQ ID NO: 28 - DNA, 21 bp, Artificial Sequence
  Description of Artificial Sequence:Synthetic Oligonucleotide
gtatacgaggatcctctagag

SEQ ID NO: 29 - DNA, 25 bp, Artificial Sequence
  Description of Artificial Sequence:Synthetic Oligonucleotide
tcgactctagagagtcctcgtatac

SEQ ID NO: 30 - DNA, 51 bp, Artificial Sequence
  Description of Artificial Sequence:Synthetic Oligonucleotide
ggaattctccatatgcaccatcatcatcatcatagcgataaaattattcac

SEQ ID NO: 31 - DNA, 21 bp, Artificial Sequence
  Description of Artificial Sequence:Synthetic Oligonucleotide
cctgtacgattactgcaggtc

SEQ ID NO: 32 - DNA, 35 bp, Artificial Sequence
  Description of Artificial Sequence:Synthetic Oligonucleotide
aacaacaacgatatcagacaaaagactaggcgcca

SEQ ID NO: 33 - DNA, 38 bp, Artificial Sequence
  Description of Artificial Sequence:Synthetic Oligonucleotide
aacaacggatccaaccaacttatatagttgctgcacca

SEQ ID NO: 34 - DNA, 26 bp, Artificial Sequence
  Description of Artificial Sequence:Synthetic Oligonucleotide
gatccatggtcgactcgagaccggtg

SEQ ID NO: 35 - DNA, 26 bp, Artificial Sequence
  Description of Artificial Sequence:Synthetic Oligonucleotide
aattcaccggtctcgagtcgaccatg

SEQ ID NO: 36 - DNA, 53 bp, Artificial Sequence
  Description of Artificial Sequence:Synthetic Oligonucleotide
gatctgaagacaacatgatcagacaaaagactaggcgccaacaacaaggatcc

SEQ ID NO: 37 - DNA, 53 bp, Artificial Sequence
  Description of Artificial Sequence:Synthetic Oligonucleotide
tcgaggatccttgttgttggcgcctagtcttttgtctgatcatgttgtcttca

SEQ ID NO: 38 - DNA, 43 bp, Artificial Sequence
  Description of Artificial Sequence:Synthetic Oligonucleotide
tgcggaatgtgtgcgaacgtggatgactgaatggatccggtac

SEQ ID NO: 39 - DNA, 35 bp, Artificial Sequence
  Description of Artificial Sequence:Synthetic Oligonucleotide
cggatccattcagtcatccacgttcgcacacattc

SEQ ID NO: 40 - DNA, 114 bp, Artificial Sequence
  Description of Artificial Sequence:Synthetic Oligonucleotide
gtcatttccactcatatgaaagtttccgagacccaaaaaaccgaaactttcgtttctagatttgccccggacgaacccagaaagggttccgacgttctcgtggaggccctcgaa

SEQ ID NO: 41 - DNA, 114 bp, Artificial Sequence
  Description of Artificial Sequence:Synthetic Oligonucleotide
ttcgagggcctccacgagaacgtcggaaccctttctgggttcgtccggggcaaatctagaaacgaaagtttcggttttttgggtctcggaaactttcatatgagtggaaatgac

SEQ ID NO: 42 - DNA, 2702 bp, Nicotiana plumbaginifolia
gaattcaccttcggagggaaccagctactagacggttcgattagtctttcgcccctatacccaagtcagacgaacgatttgcacgtcagtatcgcctgcggcctccaccagagtttcctctctctgccccgctcaggcatagttcaccatctttcgggtcccgacaggtatgctcacactcgaacccttcacagaagatcaaggtcggtcggcggtgcacctcagggggatcccaccaatcagcttccttacgcttacgggtttactcgcccgttgactcgcacacatgcggaattccggtcaggttaaggtaattgctaaattggttcatccaaatctttaaaccgttcgacgccgctttctccaatccgccgcacagccagtgttgtccatcttctaacaccgccttgtacattatactcgaaccactagctcccaaaatataagcagatgctttaagcaatgtctctcccagctctcattcaacaataatggcggcggcggctgctccatctccctcttcttccgctttctccaaagccctaatgtcctcctcctccaaatcctccaccctcctccctagatccacttttcctttcccccaccacccccacaaaaccaccccaccacccctccacctcacccccacccacattcacagccaacgccgtcgtttcaccatctccaatgtcatttccactacccaaaaagtttccgagacccaaaaaaccgaaactttcgtttcccgttttgccccggacgaacccagaaagggttccgacgttctcgtggaggccctcgaaagagaaggggttacggacgtttttgcgtacccaggcggcgcttccatggagattcaccaagctttgactcgttcaagcatcatccgcaacgtgctgccgcgtcacgagcagggtggtgtcttcgccgctgagggttacgcacgcgccaccggcttccccggcgtttgcattgctacctccggccccggtgccaccaatctcgtcagtggcctcgcggacgccctactggatagcgtccccattgttgctataaccggtcaagtgccacgtaggatgatcggtactgatgcttttcaggaaactccgattgttgaggtaactagatcgattaccaagcataattatctcgttatggacgtagaggatattcctagggttgtacgtgaggcttttttccttgcgagatcgggccggcctggccctgttttgattgatgtacctaaggatattcagcaacaattggtgatacccaactgggatcagccaatgagattgcccggttacatgtctaggttgccgaaattgcccaatgagatgcttttagaacaaattgttaggcttatttctgagtcaaagaaacctgttttgtatgtggggggtgggtgttcgcaatcgagtgaggagttgagacgattcgtggagctcacgggtatccccgtggcaagtactttgatgggtcttggagcatttccaactggggatgagctttccctttcaatgttgggtatgcatggtactgtttatgctaattatgctgtggatagtagtgatttgttgctcgcatttggggtgaggtttgatgatagagttactggaaagttagaagcttttgctagccgagcgaaaattgttcacattgatattgattcagccgagattggaaagaacaagcagcctcatgtttccatttgtgcggatataaagttggcattacagggtttgaattcgatattggagagtaaggaaggcaaactgaagctggatttttctgcttggaggcaggagttgaccgagcagaaagtgaagtacccattgaattttaaaacttttggtgatgctattcctccgcaatatgctatccaggttctagatgagttaactaatgggaatgctattataagtactggagtgggtcaacaccagatgtgggctgctcaatactataagtacagaaagccacgccaatggttgacgtctggtggattaggagcgatgggatttggtttgcccgctgctattggtgcggctgttggaagaccggatgaaattgtggttgacattgatggcgatggcagtttcatcatgaatgtgcaggagctagcaactattaaggtggagaatctcccagttaagattatgttactgaataatcaacacttgggaatggtggttcaatgggaggatcggttctacaaggctaacagagcacacacgtacctggggaatccttctaatgaggcggagatctttcctaatatgttgaaatttgcagaggcttgtggcatacctgctgcgagagtgacacacagggatgatctgagagcggctattcaaaagatgttagacactcctgggccatacttgttggatgtgattgtacctcatcaggaacatgttctacctatgattcccagtggcggggctttcaaagatgtgatcacagagggtgacgggagaagttcctattgactttgagaagctacagagctaattctaggccttgtattatctaaaataaacttctattaagccaaagatgttttgtctattagtttgttattagtttttgccgtggctttgctcgttgtcactgttgtactattaaatagttgatatttatgtttgctttaagtttgcatcaaaaaaaaaaaaaaaaaaaaaaaaaaaaaaaaactcgag

SEQ ID NO: 43 - protein, 666 aa, Nicotiana plumbaginifolia
MAAAAAPSPSSSAFSKALMSSSSKSSTLLPRSTFPFPHHPHKTTPPPLHLTPTHIHSQRRRFTISNVISTTQKVSETQKTETFVSRFAPDEPRKGSDVLVEALEREGVTDVFAYPGGASMEIHQALTRSSIIRNVLPRHEQGGVFAAEGYARATGFPGVCIATSGPGATNLVSGLADALLDSVPIVAITGQVPRRMIGTDAFQETPIVEVTRSITKHNYLVMDVEDIPRVVREAFFLARSGRPGPVLIDVPKDIQQQLVIPNWDQPMRLPGYMSRLPKLPNEMLLEQIVRLISESKKPVLYVGGGCSQSSEELRRFVELTGIPVASTLMGLGAFPTGDELSLSMLGMHGTVYANYAVDSSDLLLAFGVRFDDRVTGKLEAFASRAKIVHIDIDSAEIGKNKQPHVSICADIKLALQGLNSILESKEGKLKLDFSAWRQELTEQKVKYPLNFKTFGDAIPPQYAIQVLDELTNGNAIISTGVGQHQMWAAQYYKYRKPRQWLTSGGLGAMGFGLPAAIGAAVGRPDEIVVDIDGDGSFIMNVQELATIKVENLPVKIMLLNNQHLGMVVQWEDRFYKANRAHTYLGNPSNEAEIFPNMLKFAEACGIPAARVTHRDDLRAAIQKMLDTPGPYLLDVIVPHQEHVLPMIPSGGAFKDVITEGDGRSSY